(19) United States
(12) Patent Application Publication
(10) Pub. No.: US 2025/0115666 A1
(43) Pub. Date: Apr. 10, 2025

(54) CELL CULTURE METHODS FOR PRODUCING THERAPEUTIC PROTEINS

(71) Applicant: BRISTOL-MYERS SQUIBB COMPANY, Princeton, NJ (US)

(72) Inventors: Qin HE, Littleton, MA (US); Jianlin XU, Littleton, MA (US); Matthew Stephen REHMANN, Amherst, MA (US); Jun ` TIAN, Westford, MA (US); Michael Christopher BORYS, Groton, MA (US)

(73) Assignee: BRISTOL-MYERS SQUIBB COMPANY, Princeton, NJ (US)

(21) Appl. No.: 18/294,609

(22) PCT Filed: Aug. 4, 2022
(86) PCT No.: PCT/US2022/074507

§ 371 Date: Feb. 2, 2024

Related U.S. Application Data
(60) Provisional application No. 63/305,236, filed on Jan. 31, 2022.
(60) Provisional application No. 63/229,654, filed on Aug. 5, 2021.

(51) Int. Cl. C07K 16/28

(52) U.S. Cl. CPC C07K 16/2818; C07K 2317/14

(57) ABSTRACT

The present invention generally relates to a method of producing a therapeutic protein of interest from host cells through a new re-cloning strategy.

### CROSS-REFERENCE TO RELATED APPLICATIONS

This application claims priority to U.S. Provisional Application Ser. No. 63/229,654, filed Aug. 5, 2021 and U.S. Provisional Application Ser. No. 63/305,236, filed Jan. 31, 2022, the entire contents of which is incorporated herein by reference.

### FIELD OF THE INVENTION

The present invention generally relates to cell culture methods for producing a therapeutic protein of interest from host cells.

### BACKGROUND OF THE INVENTION

Therapeutic proteins become more popular in treatment of multiple human diseases. A major challenge facing the biopharmaceutical industry is to reduce the cost of biologics manufacturing to ensure that biologics can reach more patients. Therefore, an improved cell culture method to increase the therapeutic protein productivity with a higher titer needs to be developed.

Manufacturing titer can be improved by improving specific productivities (Qp) or increasing viable cell densities (VCD), especially through cell line development, or medium and process optimization.

Clonality of the production cell line is important for mAb product safety and efficacy in biologics manufacturing and should be assured during clone selection in alignment with the requirements of regulatory agencies (e.g., ICH Q5D Section 2.1.3, 1998; Fyre, et al., Biologicals 44 (2): 117-122 (2016)). However, re-cloning to improve upstream productivity after an early phase manufacturing process has not yet been developed.

### SUMMARY OF THE INVENTION

In certain embodiments of this invention, this disclosure provides a method of producing a recombinant polypeptide of interest, comprising: a) culturing a monoclonal cell line under optimized culture conditions; b) re-cloning the single cell of the monoclonal cell line; c) selecting the re-cloned cells with higher titer; and d) culturing the selected re-cloned cells, thereby producing the recombinant polypeptide of interest.

In certain embodiments of this invention, this disclosure provides a method of increasing titer of a recombinant polypeptide of interest, comprising: a) culturing a monoclonal cell line under optimized culture conditions; b) re-cloning the single cell of the monoclonal cell line; c) selecting the re-cloned cells with higher titer; and d) culturing the selected re-cloned cells, thereby producing the recombinant polypeptide of interest.

In certain aspects of the invention, the monoclonal cell in step a) has been cloned and selected with a selection agent, and the re-cloning in step b) is in presence of the same selection agent as used in step a).

In certain aspects of the invention, the selection agent concentration used in re-cloning in step b) is higher than the selection agent concentration used in step a).

In certain aspects of the invention, the selection agent is methionine sulfoximine (MSX).

In certain aspects of the invention, the MSX concentration in re-cloning is at least about 1 μM, at least about 2 μM, at least about 5 μM, at least about 10 μM, at least about 15 μM, at least about 20 μM, at least about 25 μM, at least about 30 μM, at least about 40 μM, at least about 50 μM, at least about 100 μM, or at least about 200 μM.

In certain preferred aspects of the invention, the MSX concentration is between about 25 μM to about 75 μM in the re-cloning step.

In certain aspects of the invention, the re-cloned cells are cultured in a bioreactor.

In certain aspects of the invention, the bioreactor is at least 10 L, at least 50 L, at least 100 L, at least 200 L, at least 500 L, at least 1,000 L, at least 2,000 L, at least 5,000 L, at least 10,000 L, at least 15,000 L, at least 20,000 L or at least 25,000 L.

In certain aspects of the invention, the monoclonal cells are from a research cell bank (RCB), a master cell bank (MCB) or a working cell bank (WCB).

In certain aspects of the invention, the cells are mammalian cells.

In certain aspects of the invention, the mammalian cells are selected from the group consisting of CHO, VERO, BHK, HEK, HeLa, COS, MDCK and hybridoma cells.

In certain aspects of the invention, the cells are CHO cells.

In certain aspects of the invention, the CHO cells are CHO glutamine synthetase (GS) knockout (GS−/−) cell line

In certain aspects of the invention, the recombinant polypeptide of interest is an antibody or antigen-binding fragment.

In certain aspects of the invention, the antibody is an mAb.

In certain aspects of the invention, the antibody or antigen-binding fragment binds an antigen selected from the group consisting of PD-1, PD-L1, CTLA-4, LAG-3, TIGIT, TIM-3, GITR, CXCR4, CD73, HER2, VEGF, CD20, CD40, CD11a, tissue factor (TF), PSCA, IL-8, IL-13, and SARS-COV-2 spike protein.

### BRIEF DESCRIPTION OF THE DRAWINGS

FIG. 1A and FIG. 1B show Southern blot integration and structure profiles for mAb1 parental and re-clone, for both the heavy chain (HC) (FIG. 1A) and light chain (LC) (FIG. 1B) at 1 week (wk) after RCB thaw and 6 weeks after RCB thaw. The untransfected parental cells (PC) are shown for comparison.

FIG. 2A and FIG. 2B show seed culture doubling times for the parental clone and the re-clone for mAb1, mAb2, mAb3 and mAb4 in the shake flask seed cultures (FIG. 2A), and doubling times in the N−1 5-L cultures (FIG. 2B). The values are reported as average±standard deviation (n≥3) or average±difference/2 (n=2).

FIG. 3A, FIG. 3B, FIG. 3C, FIG. 3D, FIG. 3E, FIG. 3F, FIG. 3G, FIG. 3H, FIG. 3I, FIG. 3J, FIG. 3K, and FIG. 3L show mAb1 culture performance, metabolite and quality attribute profiles for the parental clone and the re-clone. (FIG. 3A) N−1 fed-batch 5-L bioreactor viable cell density (VCD), (FIG. 3B) N−1 viability, (FIG. 3C) production 5-L bioreactor VCD, (FIG. 3D) production viability, (FIG. 3E) normalized titer, (FIG. 3F) normalized Qp, (FIG. 3G) production glucose profiles, (FIG. 3H) production lactate profiles, (FIG. 3I) production glutamine profiles, (FIG. 3J) production glutamate profiles, (FIG. 3K) production ammonium profiles, (FIG. 3L) in-process quality attribute profiles on day 14 at harvest. The parental day 14 titer was normalized as 1. The values are reported as average #standard deviation (n=3) or average±difference/2 (n=2).

FIG. 4A and FIG. 4B show Southern blot integration and structure profiles for mAb2 parental and re-clone, for both the heavy chain (HC) (FIG. 4A) and light chain (LC) (FIG. 4B) at 5 passages (P5) after RCB thaw and 13 passages (P13) after RCB thaw. The untransfected parental cells (PC) are shown for comparison.

FIG. 5A, FIG. 5B, FIG. 5C, FIG. 5D, FIG. 5E, FIG. 5F, FIG. 5G, FIG. 5H, FIG. 5I, FIG. 5J, FIG. 5K, and FIG. 5L show mAb2 culture performance, metabolite and quality attribute profiles for the parental clone and the re-clone. (FIG. 5A) N−1 perfusion 5-L bioreactor viable cell density (VCD), (FIG. 5B) N−1 viability, (FIG. 5C) production 5-L bioreactor VCD, (FIG. 5D) production viability, (FIG. 5E) normalized titer, (FIG. 5F) normalized Qp, (FIG. 5G) production glucose profiles, (FIG. 5H) production lactate profiles, (FIG. 5I) production glutamine profiles, (FIG. 5J) production glutamate profiles, (FIG. 5K) production ammonium profiles, (FIG. 5L) in-process quality attribute profiles on day 14 at harvest. The parental day 14 titer was normalized as 1. The values are reported as average±standard deviation (n=4).

FIG. 6A and FIG. 6B show Southern blot integration and structure profiles for mAb3 parental and re-clone, for both the heavy chain (HC) (FIG. 6A) and light chain (LC) (FIG. 6B) at 4 passages (P4) after RCB thaw and 13 passages (P13) after RCB thaw.

FIG. 7A, FIG. 7B, FIG. 7C, FIG. 7D, FIG. 7E, FIG. 7F, FIG. 7G, FIG. 7H, FIG. 7I, FIG. 7J, FIG. 7K, and FIG. 7L show mAb3 culture performance, metabolite and quality attribute profiles for the parental clone and the re-clone. (FIG. 7A) N−1 perfusion 5-L bioreactor viable cell density (VCD), (FIG. 7B) N−1 viability, (FIG. 7C) production 5-L bioreactor VCD, (FIG. 7D) production viability, (FIG. 7E) normalized titer, (FIG. 7F) normalized Qp, (FIG. 7G) production glucose profiles, (FIG. 7H) production lactate profiles, (FIG. 7I) production glutamine profiles, (FIG. 7J) production glutamate profiles, (FIG. 7K) production ammonium profiles, (FIG. 7L) in-process quality attribute profiles on day 10 at harvest. The parental day 10 titer was normalized as 1. The values are reported as average±standard deviation (n=4) or average±difference/2 (n=2).

FIG. 8A, FIG. 8B, FIG. 8C, and FIG. 8D show cell culture performance, productivity and quality attributes profiles among 5-L, 500-L and 2000-L for the re-clone producing mAb3. (FIG. 8A) production 5-L, 500-L and 2000-L bioreactor VCD, (FIG. 8B) production viability, (FIG. 8C) normalized titer, (FIG. 8D) in-process quality attribute profiles on day 10 at harvest.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, and FIG. 9L show mAb4 culture performance, metabolite and quality attribute profiles for the parental clone and the re-clone. (FIG. 9A) N−1 perfusion 5-L bioreactor viable cell density (VCD), (FIG. 9B) N−1 viability, (FIG. 9C) production 5-L bioreactor VCD, (FIG. 9D) production viability, (FIG. 9E) normalized titer, (FIG. 9F) normalized Qp, (FIG. 9G) production glucose profiles, (FIG. 9H) production lactate profiles, (FIG. 9I) production glutamine profiles, (FIG. 9J) production glutamate profiles, (FIG. 9K) production ammonium profiles, (FIG. 9L) in-process quality attribute profiles on day 14 at harvest. The parental day 14 titer was normalized as 1. The values are reported as average±standard deviation (n≥3) or average±difference/2 (n=2).

FIG. 10A and FIG. 10B show stability profiles of cell culture productivity (FIG. 10A) and quality (FIG. 10B) for the cell culture process using the mAb2 re-clone at WCB-age (i.e., the same age that the cells would be after the standard scale-up train for the upstream process from the WCB), 30 generations after WCB-age (WCB-G30) and 48 generations after WCB-age (WCB-G48).

FIG. 11A and FIG. 11B show stability profiles of cell culture productivity (FIG. 11A) and quality (FIG. 11B) for the cell culture process using the mAb3 re-clone at WCB-age (i.e., the same age that the cells would be after the standard scale-up train for the upstream process from the WCB), 15 generations after WCB-age (WCB-G15), 31 generations after WCB-age (WCB-G31), and 49 generations after WCB-age (WCB-G49). The stability study for mAb3 was conducted using the ambr15 high-throughput bioreactor system and titers were measured with the Cedex BioHT.

FIG. 12A, FIG. 12B, FIG. 12C, and FIG. 12D show cell culture performance, productivity and quality attribute profiles between 5-L and 500-L for the re-clone producing mAb1. (FIG. 12A) production 5-L and 500-L bioreactor VCD, (FIG. 12B) production viability, (FIG. 12C) normalized titer, (FIG. 12D) in-process quality attribute profiles on day 14 at harvest.

FIG. 13A, and FIG. 13B show clonal titer distribution profiles for mAb1 parental clone and re-clone screening in 96 well plates (day 7 normalized titers, FIG. 13A) and C50 SpinTtube bioreactors (day 14 normalized titers, FIG. 13B). The titers were normalized to the highest clonal titer of the parental or re-clone screening.

FIG. 14A, and FIG. 14B show clonal titer distribution profiles for mAb2 parental clone and re-clone screening in 96 well plates (day 7 normalized titers, FIG. 14A) and C50 SpinTtube bioreactors (day 14 normalized titers, FIG. 14B). The titers were normalized to the highest clonal titer of the parental or re-clone screening.

FIG. 15A, and FIG. 15B show clonal titer distribution profiles for mAb3 parental clone and re-clone screening in 96 well plates (day 7 normalized titers, FIG. 15A) and C50 SpinTtube bioreactors (day 14 normalized titers, FIG. 15B). The titers were normalized to the highest clonal titer of the parental or re-clone screening.

### DETAILED DESCRIPTION OF THE INVENTION

The present invention relates to a method of producing a recombinant polypeptide of interest, comprising: a) culturing a monoclonal cell line under optimized culture conditions; b) re-cloning the single cell of the monoclonal cell line; c) selecting the re-cloned cells with higher titer; and d) culturing the selected re-cloned cells, thereby producing the recombinant polypeptide of interest.

In certain embodiments of this invention, this disclosure provides a method of increasing titer of a recombinant polypeptide of interest, comprising: a) culturing a monoclonal cell line under optimized culture conditions; b) re-cloning the single cell of the monoclonal cell line; c) selecting the re-cloned cells with higher titer; and d) culturing the selected re-cloned cells, thereby producing the recombinant polypeptide of interest.

In certain aspects of the invention, the monoclonal cell in step a) has been cloned and selected with a selection agent, and the re-cloning in step b) is in presence of the same selection agent as used in step a).

In certain embodiments of this invention, this disclosure provides a method of producing a recombinant polypeptide of interest, comprising: a) culturing a monoclonal cell line under optimized culture conditions, wherein the monoclonal cell has been cloned and selected with a selection agent; b) re-cloning the single cell of the monoclonal cell line in the presence of a selection agent; c) selecting the re-cloned cells with higher titer; and d) culturing the selected re-cloned cells, thereby producing the recombinant polypeptide of interest.

In certain embodiments of this invention, this disclosure provides a method of increasing titer of a recombinant polypeptide of interest, comprising: a) culturing a monoclonal cell line under optimized culture conditions, wherein the monoclonal cell has been cloned and selected with a selection agent; b) re-cloning the single cell of the monoclonal cell line in the presence of a selection agent; c) selecting the re-cloned cells with higher titer; and d) culturing the selected re-cloned cells, thereby producing the recombinant polypeptide of interest.

### Definitions

In order that the present disclosure may be more readily understood, certain terms are first defined. As used in this application, except as otherwise expressly provided herein, each of the following terms shall have the meaning set forth below. Additional definitions are set forth throughout the application.

The indefinite articles “a” or “an” should be understood to refer to “one or more” of any recited or enumerated component.

The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean a range of plus or minus 50% of a stated reference value, preferably a range of plus or minus 25%, or more preferably a range of plus or minus 10%. When particular values or compositions are provided in the application and claims, unless otherwise stated, the meaning of “about” should be assumed to be within an acceptable error range for that particular value or composition.

A “protein” as used herein refers to a peptide-linked chain of amino acids regardless of length. One or more amino acid residues in the protein may contain a modification such as, but not limited to, glycosylation, phosphorylation or disulfide bond formation. The term “protein” is used interchangeably herein with “polypeptide.”

The term “protein of interest” as used herein is used in its broadest sense to include any protein (either natural or recombinant), present in a mixture, for which purification is desired. Such proteins of interest include, without limitation, enzymes, hormones, growth factors, cytokines, immunoglobulins (e.g., antibodies), and/or any fusion proteins.

The term “therapeutic protein,” as used herein, refers to any protein that is known to be useful for the prevention, treatment, or amelioration of a disease or disorder, e.g., an antibody, growth factor, cell surface receptor, cytokine, hormone, toxin, or fragments and/or fusion proteins of any of the foregoing.

An “antibody” (Ab) shall include, without limitation, a glycoprotein immunoglobulin (Ig) which binds specifically to an antigen and comprises at least two heavy (H) chains and two light (L) chains interconnected by disulfide bonds, or an antigen-binding portion thereof.

The term “monoclonal antibody” (mAb) as used herein refers to a non-naturally occurring preparation of Ab molecules of single molecular composition, i.e., Ab molecules whose primary sequences are essentially identical and which exhibit a single binding specificity and affinity for a particular epitope. An mAb is an example of an isolated Ab. MAbs may be produced by hybridoma, recombinant, transgenic or other techniques known to those skilled in the art.

The term “mAb of interest” as used herein refers to an mAb, present in a mixture, for which purification is desired.

A “chimeric” Ab as used herein refers to an Ab in which the variable regions are derived from one species and the constant regions are derived from another species, such as an Ab in which the variable regions are derived from a mouse Ab and the constant regions are derived from a human Ab.

A “human” mAb (HuMAb) as used herein refers to an mAb having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human” Ab, as used herein, is not intended to include Abs in which CDR sequences derived from the germline of another mammalian species, such as a mouse, have been grafted onto human framework sequences. The terms “human” Abs and “fully human” Abs are used synonymously.

A “humanized” mAb as used herein refers to an mAb in which some, most or all of the amino acids outside the CDR domains of a non-human mAb are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

In one embodiment of the present invention, the mAb is a chimeric mAb, a human mAb or a humanized mAb.

An “anti-antigen” Ab as used herein refers to an Ab that binds specifically to an antigen. For example, an anti-PD-1 Ab is an Ab that binds specifically to PD-1.

The term “culture”, “cell culture” or “mammalian cell culture” as used herein refers to a mammalian cell population that is suspended in a medium under conditions suitable to survival and/or growth of the cell population. As will be clear to those of ordinary skill in the art, these terms as used herein may refer to the combination comprising the mammalian cell population and the medium in which the population is suspended.

The term “culturing” or “cell culturing” refers to a maintenance or growth process of a mammalian cell in a liquid culture medium under a controlled set of physical conditions.

The term “seeding”, “seeded”, “inoculation”, “inoculating” or “inoculated” as used herein refers to a process of providing a cell culture to a bioreactor or another vessel. The cells may have been propagated previously in another bioreactor or vessel. Alternatively, the cells may have been frozen and thawed immediately prior to providing them to the bioreactor or vessel. The term refers to any number of cells, including a single cell.

The term “cloning” or “single cell cloning” or “cell cloning”, in connection with cell culture as used herein refers to a process wherein single cells can be identified and isolated from a cell pool with cells of different genotypes, and then replicated to form a cell population consisting of a plurality of genetically identical cells.

The term “recloning” or “re-cloning”, in connection with cell culture as used herein refers to a process wherein single cells can be further identified and isolated from “a monoclonal cell line/culture” The cells from the monoclonal cell line/culture are deposited individually, i.e. only one (1) cell per culture vessel, and then expanded to form a cell population of identical cells.

The term “a monoclonal cell line” or “a monoclonal cell culture” as used herein refers to a cell population consisting of identical cells which originate from one (1) single cell. If during the cultivation, spontaneous changes in the genome, for example mutations and/or translocations occur, the culture still is regarded as a monoclonal cell line.

The term “selective agent” as used herein refers to a molecule, or a set of culture conditions that are lethal or inhibitory to host cells in the absence of a selectable marker. Only those cells expressing a selectable marker survive or have the growth advantage in the presence of the selective agent. One of ordinary skill in the art will be aware of and will be able to choose suitable selective agent for use in practicing the present invention.

In certain aspects of the invention, the selection agent concentration used in recloning step is higher than the selection agent concentration used in cloning/maintaining parental monoclonal cell line.

In certain aspects of the invention, the selection agent is methionine sulfoximine (MSX).

In certain aspects of the invention, the MSX concentration in re-cloning is at least about 1 μM, at least about 2 μM, at least about 5 μM, at least about 10 μM, at least about 15 μM, at least about 20 μM, at least about 25 μM, at least about 30 μM, at least about 40 μM, at least about 50 μM, at least about 100 μM, or at least about 200 μM.

In certain aspects of the invention, the MSX concentration is between about 25 μM to about 75 μM in re-cloning step.

The term “master cell bank” or “MCB” as used herein refers to an aliquot of a single pool of cells that generally has been prepared from the selected cell clone under defined conditions, dispensed into multiple containers, and stored under defined conditions. The MCB is used to derive all working cell banks (WCB). The WCB is prepared from aliquots of a homogeneous suspension of cells obtained from culturing the MCB under defined culture conditions. A monoclonal cell line of single-cell origin is created to establish each master cell bank (MCB) while running processes that comply with good manufacturing practices (GMPs) and generating clinical trial materials.

The term “research cell bank” or “RCB” as used herein refers to a cryopreserved bank of selected monoclonal cell line, not fully characterized but suitable for use as a seed stock for the development of a fully characterized master cell bank.

In certain aspects of the invention, the monoclonal cells are from a research cell bank (RCB), a master cell bank (MCB) or a working cell bank (WCB).

The term “basal medium” as used herein refers to a solution containing nutrients which nourish growing mammalian cells. Typically, the solution provides essential and non-essential amino acids, vitamins, energy sources, lipids, and trace elements required by the cell for minimal growth and/or survival. The solution may also contain components that enhance growth and/or survival above the minimal rate, including hormones and growth factors. The solution is preferably formulated to a pH and salt concentration optimal for cell survival and proliferation. Various components may be added to a basal medium to benefit cell growth. The medium may also be a “chemically-defined medium” that does not contain serum, hydrolysates or components of unknown composition. Defined media are free of animal-derived components and all components have a known chemical structure.

The term “batch” or “batch culture” as used herein refers to a method of culturing cells in which all the components that will ultimately be used in culturing the cells, including the basal medium as well as the cells themselves, are provided at the beginning of the culturing process. A batch culture is typically stopped at some point and the cells and/or components in the medium are harvested and optionally purified.

The term “fed-batch” or “fed-batch culture” means the incremental or continuous addition of a feed medium to an initial cell culture without substantial or significant removal of the initial basal medium from the cell culture. In some instances, the feed medium is the same as the initial basal medium. In some instances, the feed medium is a concentrated form of the basal medium. In some instances, a feed medium may be different than the base medium but contain the desired nutritional supplements.

The term “perfusion” or “perfusion process” as used herein refers to a method of culturing cells in which equivalent volumes of media (containing nutritional supplements) are simultaneously added and removed from the bioreactor while the cells are retained in the reactor. A volume of cells and media corresponding to the supplement media is typically removed on a continuous or semi-continuous basis and is optionally purified. Typically, a cell culture process involving a perfusion process is referred to as “perfusion culture.” A fresh medium may be identical, similar to, or different than the base medium used in the cell culture process.

The term “N−1 stage” as used herein refers to the last seed expansion stage right before production inoculation. The N−1 stage is the final cell growth step before seeding the production bioreactor for production of a protein of interest. The terms “N−2 stage” and “N−3 stage” as used herein refers to the period of time during cell growth and expansion and, typically, before inoculation of N production stage. The N−3 stage is the cell growth stage used to increase viable cell density to be used in the N−2 stage. The N−2 stage is the cell growth stage used to increase viable cell density to be used in the N−1 stage.

The term “production stage” or “N production stage” of the cell culture refers to the last stage of cell culture. During the production stage, the cells are cultured with a primary focus of producing a protein of interest, for example an mAb. The production stage is commonly referred to as “N” or last stage of cell culture manufacturing.

The term “bioreactor” as used herein refers to any vessel used for the growth of a mammalian cell culture. The bioreactor can be of any size so long as it is useful for the culturing of mammalian cells. Typically, the bioreactor will be at least 1 liter and may be 2, 5, 10, 50, 100, 250, 500, 1000, 2500, 5000, 8000, 10,000, 12,000, 15,000, 20,000, 25,000 liters or more, or any volume in between. The internal conditions of the bioreactor, including, but not limited to pH and temperature, are typically controlled during the culturing period. The bioreactor can be composed of any material that is suitable for holding mammalian cell cultures suspended in media under the culture conditions of the present invention, including glass, plastic or metal.

In certain aspects of the invention, the re-cloned cells are cultured in a bioreactor.

In certain aspects of the invention, the bioreactor is at least 10 L, at least 50 L, at least 100 L, at least 200 L, at least 500 L, at least 1,000 L, at least 2,000 L, at least 5,000 L, at least 10,000 L, at least 15,000 L, at least 20,000 L or at least 25,000 L.

The term “production bioreactor” as used herein refers to the final bioreactor used in the production stage of the cell culture for the production of a protein of interest (e.g. mAb). The volume of the large-scale cell culture production bioreactor is typically at least 500 liters and may be 1000, 2500, 5000, 8000, 10,000, 12,000, 15,000, 20,000, 25,000 liters or more, or any volume in between. One of ordinary skill in the art will be aware of and will be able to choose suitable bioreactors for use in practicing the present invention.

In certain aspects of the invention, the re-cloned cells are cultured in a production bioreactor.

In certain aspects of the invention, the production bioreactor is at least 500 L, at least 1,000 L, at least 2,000 L, at least 5,000 L, at least 10,000 L, at least 15,000 L, at least 20,000 at least 25,000 L.

The term “titer” as used herein refers to the total amount of recombinantly expressed a protein of interest, for example, an mAb produced by a mammalian cell culture, divided by a given amount of medium volume. Titer is typically expressed in units of grams of polypeptide or protein per liter of culture medium.

In certain aspects of the invention, the titer produced from the recloned cells is improved by at least 1%, at least 5%, at least 15%, at least 20%, at least 25%, at least 30% at least 40%, at least 50%, at least 60%, at least 70%, at least 80%, at least 90%, at least 100%, at least 150%, at least 200%, at least 300% at least 400%, or at least 500% over the parental clone.

In certain aspects of the invention, the titer produced from the recloned cells is improved by at least 10% over the parental clone.

Various aspects of the invention are described in further detail in the following subsections.

### Host Cells

Any mammalian cell or cell type susceptible to cell culture, and to expression of proteins, may be utilized in accordance with the present invention. Non-limiting examples of mammalian cells that may be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells±DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL5 1); TRI cells (Mather et al., Annals N.Y. Acad. Sci., 383:44-68 (1982)); MRC 5 cells; FS4 cells; and a human hepatoma line (Hep G2). In certain aspects of the invention, the cells are selected from the group consisting of CHO, VERO, BHK, HEK, HeLa, COS, MDCK and hybridoma cells.

CHO cells are the most utilized host for biologics manufacturing, especially for mAb production. During CHO cell line development, the most common gene selection systems use dihydrofolate reductase (DHFR)-based methotrexate (MTX) or L-glutamine synthetase (GS)-based methionine sulfoximine (MSX) to amplify and maintain the gene of interest (GOI). In particular, the use of the GS-based system has become increasingly common in recent years due to several advantages, which include (i) fewer amplification steps required to achieve higher cell productivity, leading to reduced cell line development timelines compared to DHFR-based system, (ii) improved selection stringency and efficiency in GS knockout cell lines with construct promoter engineering, and (iii) GS overexpression that reduces culture ammonium levels by converting glutamate and ammonium into the essential amino acid glutamine.

The GS-based system has been used in CHO cell lines that have one endogenous GS deletion or a double knockout, introducing the exogenous GS gene in the expression construct and using medium with the selection reagent MSX that inhibits GS enzymatic activity. Relative to DHFR-based system, GS knockout cell lines (GS^{-/-}) provide sufficient selection stringency without MSX or with low MSX concentrations, while 25 μM MSX coupled with the GS-knockout cell line led to higher selection efficiency compared with CHOK1SV cell lines at higher MSX concentrations (Fan, et al., Biotechnol Bioeng., 109 (4): 1007-1015 (2012)). A previous report showed that increasing the MSX concentration in the seed train stage after clone selection increased productivity without significant impacts on cell growth, GS and target gene copy numbers and expression, and maintained product quality attributes in multiple GS knockout cell lines (Tian et al., Engineering in Life Sciences 20(3-4): 112-125 (2020)).

CHO cells exhibit genomic instability including mutations and changes in gene copy number, chromosome rearrangement (i.e., changes in chromosome structure and number), and epigenetic changes to regulate gene expression by DNA methylation or histone modifications, after prolonged culture under selection pressure (Kim et al., Biotechnol Bioeng 108(10): 2434-2446(2011); Tharmalingam et al., Biotechnol Prog 34(3): 613-623(2018); Vcelar et al., Biotechnol Bioeng 115(1): 165-173(2018); Feichtinger et al., Biotechnology and Bioengineering 113 (10): 2241-2253 (2016)). The genomic instability of CHO cells has been linked to heterogeneity of cell growth, mAb productivity and quality attributes with aged cell cultures and presents a challenge for the stability of cell line productivity and product quality attributes in biologics manufacturing processes (Davies et al., Biotechnol Bioeng 110(1): 260-274 (2013); Ko et al., Biotechnol Prog 34(3): 624-634 (2018)).

In certain aspects of the invention, the host cell is a CHO cell line. In certain aspects of the invention, the CHO cells are CHO glutamine synthetase (GS) knockout (GS^{-/-}) cell line.

### Providing a Mammalian Cell Culture

Once a cell that expresses the protein of interest has been identified, the cell is propagated in culture by any of the variety of methods well-known to one of ordinary skill in the art. The cell expressing the protein of interest is typically propagated by growing it at a temperature and in a medium that is conducive to the survival, growth and viability of the cell. The initial culture volume can be of any size, but it is often smaller than the culture volume of the production bioreactor used in the final production of the protein of interest, and frequently cells are passaged several times in bioreactors of increasing volume prior to seeding the production bioreactor. Once the cells have reached a specific viable cell density, the cells are grown in a bioreactor to further increase the number of viable cells. These bioreactors are referred to as N−1, N−2, N−3, and etc. “N” refers to the main production culture bioreactor, while the “N−1” means the bioreactor prior to the main production culture, and so forth.

Generally, cell cultures of N−1 may be grown to a desired density before seeding the next production bioreactor. It is preferred that most of the cells remain alive prior to seeding, although total or near total viability is not required.

### Production of Expressed Protein

During the production stage, the cells are cultured with a primary focus of producing a protein of interest, for example an mAb of interest. The production stage is commonly referred to as “N” or last stage of cell culture manufacturing. Generally, a production bioreactor is applied for the final bioreactor used in the production stage of the cell culture for the production of a protein of interest (e.g. mAb). The volume of the large-scale cell culture production bioreactor is typically at least 500 liters and may be 1000, 2500, 5000, 8000, 10,000, 12,000, 15,000, 20,000 liters or more, or any volume in between. One of ordinary skill in the art will be aware of and will be able to choose suitable bioreactors for use in practicing the present invention.

In certain aspects of the invention, the cell culture is a perfusion, a batch or a fed batch cell culture. In certain preferred aspects, the cell culture in a production bioreactor is a fed batch cell culture.

At the end of production bioreactor run, cell culture broth is taken out and harvested with removal of cells and cell debris to yield a clarified bulk suitable for protein purification. This is generally accomplished by any suitable technique, including but not limited to centrifugation, depth filtration and sterile filtration, depending on scale and facility capability.

### Recombinant Antibody

The methods of the present invention can be used for large-scale production of any antibody.

Antibodies within the scope of the present invention include, but are not limited to: anti-HER2 antibodies including Trastuzumab (HERCEPTIN®) (Carter et al., Proc. Natl. Acad. Sci. USA, 89:4285-4289 (1992); anti-HER3 antibodies; anti-HER4 antibodies; U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108B1, or Tositumomab (BEXXAR®); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN® (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58: 377-380 (1994)); anti-IgE (Presta et al., J. Immunol. 151:2623-2632 (1993), and International Publication No. WO 95/19181); anti-CD18 (U.S. Pat. No. 5,622,700, issued Apr. 22, 1997, or as in WO 97/26912, published Jul. 31, 1997); anti-IgE (including E25, E26 and E27; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al., J. Immunol. 156 (4): 1646-1653 (1996), and Dhainaut et al., Crit. Care Med. 23 (9): 1461-1469 (1995)); anti-Tissue Factor (TF) (European Patent No. 0 420 937 B1 granted Nov. 9, 1994); anti-human α₄β₇ integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al., Arthritis Rheum 39 (1): 52-56 (1996)); anti-CD52 antibodies such as CAMPATH-1H (Riechmann et al., Nature 332:323-337 (1988)); anti-Fc receptor antibodies such as the M22 antibody directed against FcγRI as in Graziano et al., J. Immunol. 155 (10): 4996-5002 (1995); anti-carcinoembryonic antigen (CEA) antibodies such as hMN−14 (Sharkey et al., Cancer Res. 55 (23 Suppl): 5935s-5945s (1995); antibodies directed against breast epithelial cells including huBrE-3, hu-Mc 3 and CHL6 (Ceriani et al., Cancer Res. 55 (23): 5852s-5856s (1995); and Richman et al., Cancer Res. 55 (23 Supp): 5916s-5920s (1995)); antibodies that bind to colon carcinoma cells such as C242 (Litton et al., Eur J. Immunol. 26 (1): 1-9 (1996)); anti-CD38 antibodies, e.g. AT 13/5 (Ellis et al., J. Immunol. 155 (2): 925-937 (1995)); anti-CD33 antibodies such as Hu M195 (Jurcic et al., Cancer Res 55 (23 Suppl): 5908s-5910s (1995) and CMA-676 or CDP771; anti-CD22 antibodies such as LL2 or LymphoCide (Juweid et al., Cancer Res 55 (23 Suppl): 5899s-5907s (1995)); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); anti-human leukocyte antigen (HLA) antibodies such as Smart ID10; anti-PD-1 antibodies; anti-PD-L1 antibodies; anti-LAG-3 antibodies; anti-GITR antibodies; anti-TIGIT antibodies; anti-CXCR4 antibodies; anti-CD73 antibodies; anti-IL-13 antibodies, anti-SARS-COV-2 spike protein antibodies and the anti-HLA DR antibody Oncolym (Lym-1). In certain aspects of the invention, the recombinant polypeptide of interest is an antibody or antigen-binding fragment.

In certain aspects of the invention, the antibody is an mAb. In certain aspects of the invention, the antibody or antigen-binding fragment binds an antigen selected from the group consisting of PD-1, PD-L1, CTLA-4, LAG-3, TIGIT, TIM-3, GITR, CXCR4, CD73, HER2, VEGF, CD20, CD40, CD11a, tissue factor (TF), PSCA, IL-8, IL-13, SARS-COV-2 spike protein.

In certain aspects of the invention, the antibody is Nivolumab.

The foregoing description is to be understood as being representative only and is not intended to be limiting. Alternative methods and materials for implementing the invention and also additional applications will be apparent to one of skill in the art and are intended to be included within the accompanying claims.

### Example 1

### Materials and Methods

### Cell Line, Media, Re-Clone and Seed Expansion

Four CHO GS^{-/-} cell lines were used for expression of 4 proprietary human mAbs (mAb1, mAb2, mAb3 and mAb4) generated from the same parental CHO G/S^{-/-} cell line. The four cell lines were cloned and cultured in proprietary chemically defined seed medium without L-glutamine and with 1×MSX following standard proprietary procedures developed in Bristol Myers Squibb (BMS) for early phase development and clinical manufacturing.

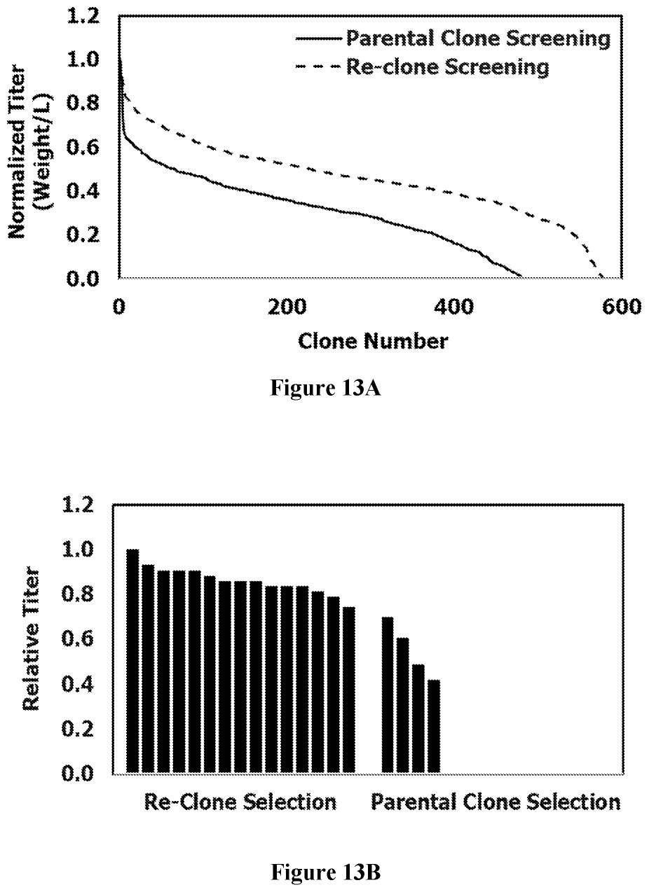

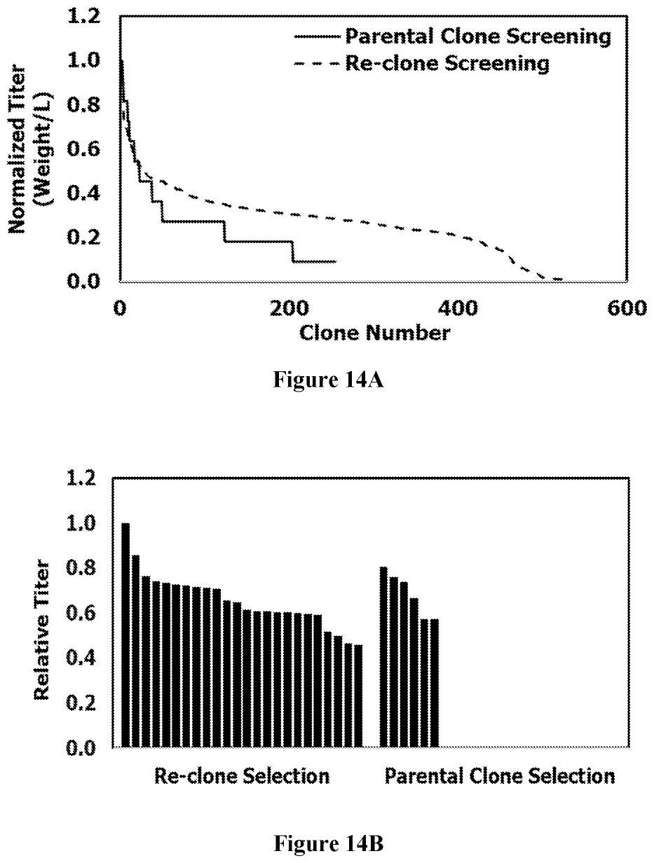

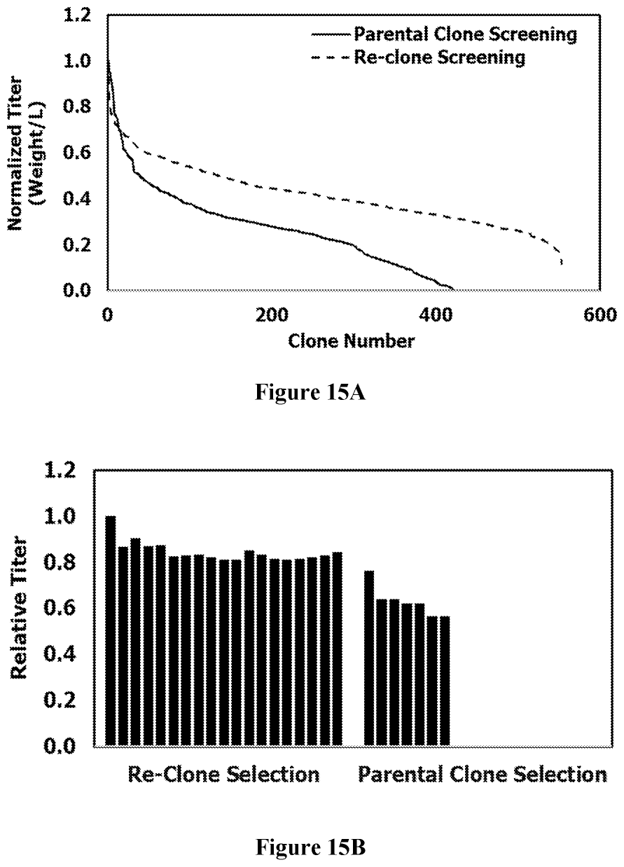

For re-cloning, three of the cell lines (producing mAb1, mAb2 and mAb3) were thawed from their parental research cell bank (RCB) or master cell bank (MCB) vials and cultured in seed medium containing 1×MSX for one or two passages (3 to 4 days per passage) and then in seed medium containing 4×MSX for up to eight passages in shake flasks. mAb4 parental MCB cell line was thawed and maintained in seed medium containing 1×MSX for eight passages. The shake flasks were cultured in a humidified incubator (Climo-Shaker, Kuhner) at 36.5° C., 5% CO₂ and 135 rpm. Single cell sorting (SCC) was performed by FACS (BD, San Jose, CA) into 96-well plates during which 1000 cells were sorted into single wells, and the cells were then cultured with seed medium containing 1× or 4×MSX in the incubator at 36.5° C. and 5% CO₂. Cellavista images were reviewed on days-1, 0, 3, 7, and day 10 to confirm clonality. With an average clonal outgrowth efficiency of 50-60%, confluency and static day 7 titers were tested on 523-577 clones (FIGS. 13-15). Following BMS standard procedures, the top 96 clones were moved forward into fed-batch studies conducted in 24 Deep Well plates, and of those, the top 24 re-clones were selected based on titers measured by Octet (ForteBio, Fremont, CA). To verify the expected sequence and search for any single amino acid substitution sequence variants, peptide mapping liquid chromatography-tandem mass spectrometry was employed.

The top 15-24 re-clones were evaluated in TubeSpin bioreactors with BMS-proprietary chemically defined basal and feed media, inoculated at 0.5×10⁶ cells/mL and cultured for 14 days at 36.5° C., 5% CO₂ and 80% humidity. Feed was added every other day, starting on day 4, at 6% of the initial culture volume. Culture glucose levels were monitored with Nova (Nova Biomedical, Waltham, MA) or a glucometer and added up to 8 g/L on even days. The titers were assessed on days 4, 6, 8, 10, 12 and 14 using the Octet and day 14 titers were confirmed by a Protein A UPLC method. Research cell banks (RCBs) were generated for the top six re-clones based on cell doubling time, production peak viable cell density (VCD), mAb titer and cell specific productivity (Qp).

The top six re-clones were evaluated with the lead upstream process conditions for the parental clones in glass 5-L bioreactors to identify and select the lead re-clone. To evaluate the top six re-clones, cells were thawed from RCB vials and cultured in shake flasks with the seed medium with 1×MSX (for mAb4) or 4×MSX (for mAb1, mAb2, and mAb3) prior to inoculation of N−1 seed bioreactors. The lead re-clones were selected based on cell doubling time, production culture peak VCD, titer, specific productivity, quality attributes, mAb gene and protein sequences, productivity and genetic stability, each of which was compared with the parental cell line.

### N−1 Seed Cultures

The N−1 cultures were run in 5-L glass vessels (Sartorius) in either fed-batch or perfusion mode. Specifically, the N−1 bioreactors were run in fed-batch mode for mAb1 re-clone selection and in perfusion mode for mAb2, mAb3 and mAb4 re-clone selection to align with the processes developed for the parental clone. The fed-batch N−1 culture was fed starting on day 2 with a BMS-proprietary feed at a fixed percentage of the initial culture volume and cultured for four days. The N−1 medium had the same composition as the inoculum medium (with 4×MSX for mAb1, mAb2 and mAb3 re-clones and 1×MSX for mAb4), except additional glucose was added in some cases to prevent depletion of glucose at the higher cell densities achieved in the N−1.

For perfusion N−1 cultures, an auxiliary alternating tangential flow filtration (ATF; Repligen) device was connected to the 5-L bioreactor to continuously perfuse spent culture medium, while fresh medium was continuously added at the same rate. Perfusion rate was controlled based on VCD as measured by an online capacitance probe (Hamilton). Perfusion was started on day 1 at a rate of 0.08 nL/cell/day for mAb2 and a rate of 0.04 nL/cell/day for mAb3 and mAb4. Temperature was maintained at 36.5° C., dissolved oxygen was maintained at 40%, and pH was controlled with a setpoint of 7.1 to 7.2 and a proprietary deadband in the N−1 cultures.

### Fed-Batch Production Cultures

Fed-batch production bioreactor runs were performed in 50-mL TubeSpin and 5-L bioreactors for up to 14 days. Basal and feed media were specified for the four mAb re-clones based on the processes developed for the parental clones. Dissolved oxygen was maintained at 40% and pH was controlled at a setpoint of pH 7.1 to 7.2 and a proprietary deadband using 1 M Na₂CO₃ and CO₂ gas in 5-L bioreators. 5% CO₂ and 80% humidity were maintained in incubator for 50-mL TubeSpin bioreactors. Temperature was initially maintained at 36.5° C., and, in some cases, shifted to a proprietary lower temperature to align with conditions developed for the parental clone.

### In-Process Cell Culture and Quality Attribute Assays

Cell culture broth was sampled from each bioreactor daily and analyzed for gases, cell count, and metabolites. Offline pH, pCO₂ and pO₂ were measured using a Bioprofile pHOx analyzer (Nova Biomedical, Waltham, MA). VCD and cell viability were quantified in a Vi-CELL XR automatic cell counter (Beckman Coulter, Jersey City, NJ). Glucose, glutamine, glutamate, lactate, and ammonia were quantified in a Cedex Bio HT (Roche CustomBiotech, Penzburg, Germany).

For titer measurements, the cell culture broth was centrifuged at 1000 g for 5 to 10 minutes, and the supernatant sample was analyzed using a Protein A UPLC method. The normalized titer was quantified as the actual titer (g/L) at each time point divided by the average of the corresponding parental clone day 14 titer (g/L) for each mAb. Overall cell specific productivity (normalized weight/cell/day) was calculated based on normalized titers (Xu et al. Bioresources and Bioprocessing 7 (1): 17, (2020))

The supernatant samples were purified by Protein A chromatography prior to quality measurements. The methods used for in-process quality attributes were similar to those described by Yongky et al, (MAbs 11 (8): 1502-1514, 2019). Charge variant species (acidic, main and basic) were measured by imaged capillary isoelectric focusing (iCIEF) or cation exchange chromatography (CEX). N-glycan profiles were measured using a commercially available RapiFluor-MS N-Glycan kit (Waters, Milford, MA). Size exclusion chromatography was used to measure high molecular weight (HMW) and low molecular weight (LMW). Copy number analysis

Copy number analysis was performed by real time qPCR analysis (Tian, 2020). Cell pellets taken from WCB-age seed cultures were used to extract genomic DNA. Primers specific to each mAb were used. The copy number was determined by extrapolating from each mAb molecular standard curve that was generated by normalizing to the GAPDH gene.

### Southern Blot Analysis

Southern blot analysis was used to determine the structural and integration pro-files of the target HC and LC genes. Genomic DNA isolated from the seed culture cell pellets was digested with the restriction enzymes specific for the target gene expression plasmids. The digested DNA was subjected to agarose gel electrophoresis and hybridization with the gene-specific hybridization probes (Tian, 2020).

### Potency Assays

Drug substances were purified from the parental clone and re-clone production day 14 samples and applied in ELISA binding potency assay for mAb1 and mAb3 or cell-based potency assay for mAb2. The potency of mAb1, mAb2 and mAb3 is determined relative to the standard curve generated using the reference standard of mAb1, mAb2 and mAb3, respectively.

### Example 2

Re-Clone Process Development for mAb1 Production

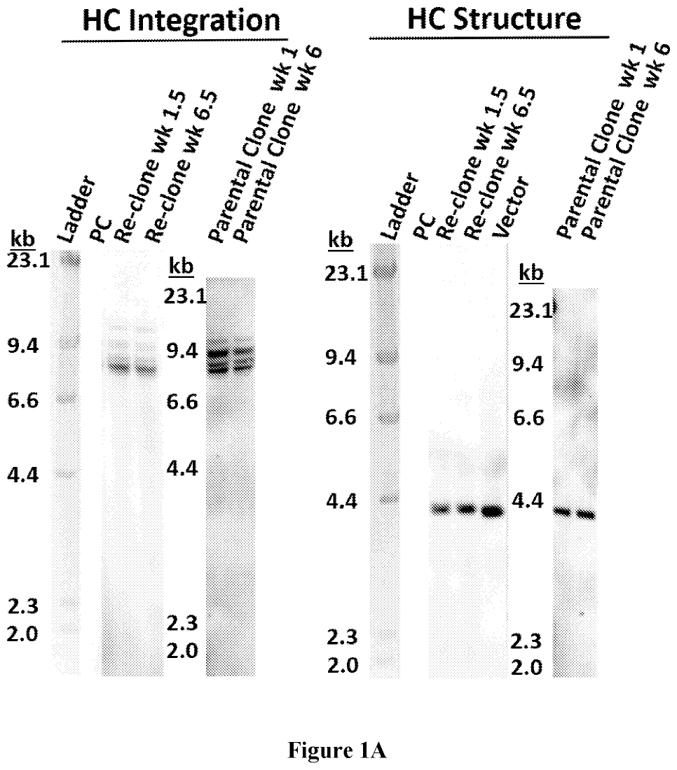

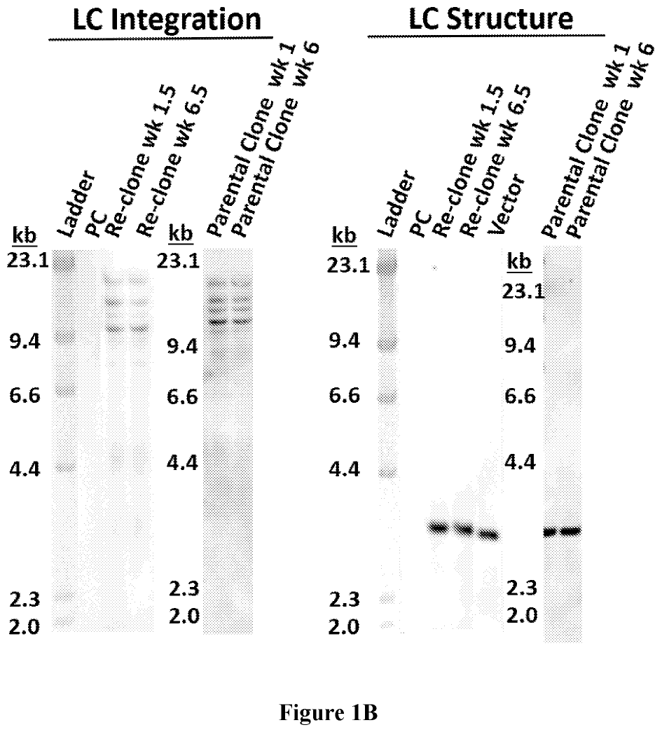

mAb1 had low productivity in first-in-human (FIH) clinical manufacturing. In an attempt to increase the productivity, the MCB was re-cloned. Specifically, an MCB vial of parental mAb1 was thawed in seed media with 1×MSX, cultured for one passage and then cultured for seven passages in seed media with 4×MSX prior to single cell sorting since internal studies demonstrated that 4×MSX in the seed train increased mAb1 production titer. After single cell sorting, new RCBs were made using the re-clones. The lead re-clone and the parental clone for mAb1 had the same mAb gene sequences, the same target gene integration and structure profiles (FIG. 1A and FIG. 1B), and similar heavy chain (HC) and light chain (LC) gene copy numbers (Table 1).

TABLE 1
Target IgG heavy chain (HC) and light chain (LC) gene copy number
(GCN) comparison for the parental clone and the re-clone for mAb1,
mAb2 and mAb3 (n = 3).

| Cell Line | Clone | HC GCN | LC GCN |
|---|---|---|---|
| mAb1 | Parental Clone | 94.9 ± 18.6 | 148.9 ± 23.7 |
| | Re-clone | 84.7 ± 10.7 | 131.0 ± 8.7 |
| mAb2 | Parental Clone | 40.5 ± 9.1 | 37.2 ± 6.8 |
| | Re-clone | 39.0 ± 1.0 | 35.7 ± 0.8 |
| mAb3 | Parental Clone | 49.4 ± 1.1 | 49.7 ± 0.4 |
| | Re-clone | 49.1 ± 1.7 | 49.6 ± 0.9 |

Compared to the parental clone with the same conditions for mAb1, the fed-batch titer for the re-clone increased by 62% in 50-mL TubeSpin bioreactors (Table 2), indicating that the re-cloning strategy did increase titer other than just increasing MSX concentrations in seed media alone for mAb1.

TABLE 2
Normalized day 14 titer comparison between the parental clone and
re-clone in 50-mL TubeSpin bioreactors for mAb1 and mAb2 using
the same media and fed-batch production conditions. The values are
reported as average ± difference/2 (n = 2).

| Cell Line | Clone | Normalized Titer |
|---|---|---|
| mAb1 | Parental Clone | 1.00 ± 0.02 |
| | Re-clone | 1.62 ± 0.09 |
| mAb2 | Parental Clone | 1.00 ± 0.01 |
| | Re-clone | 1.25 ± 0.01 |

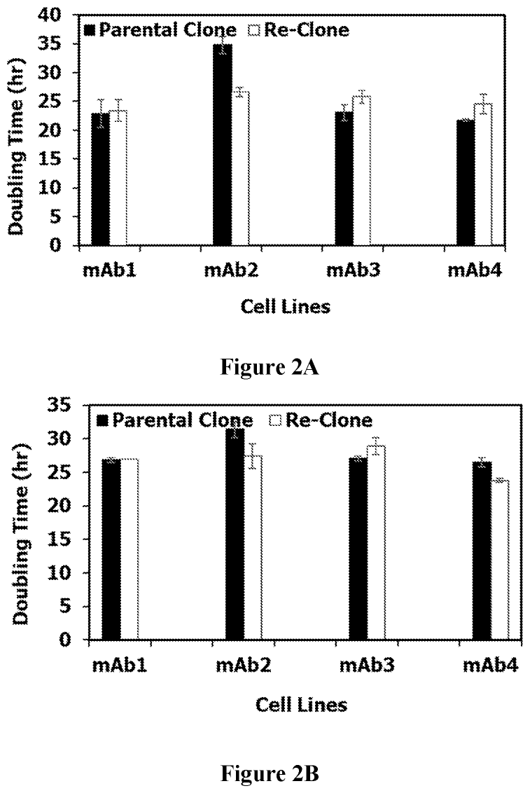

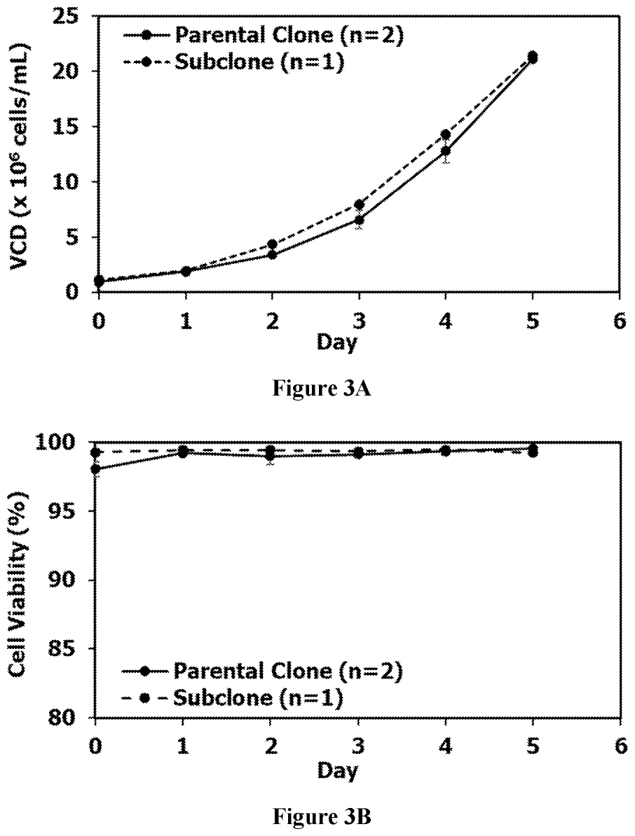

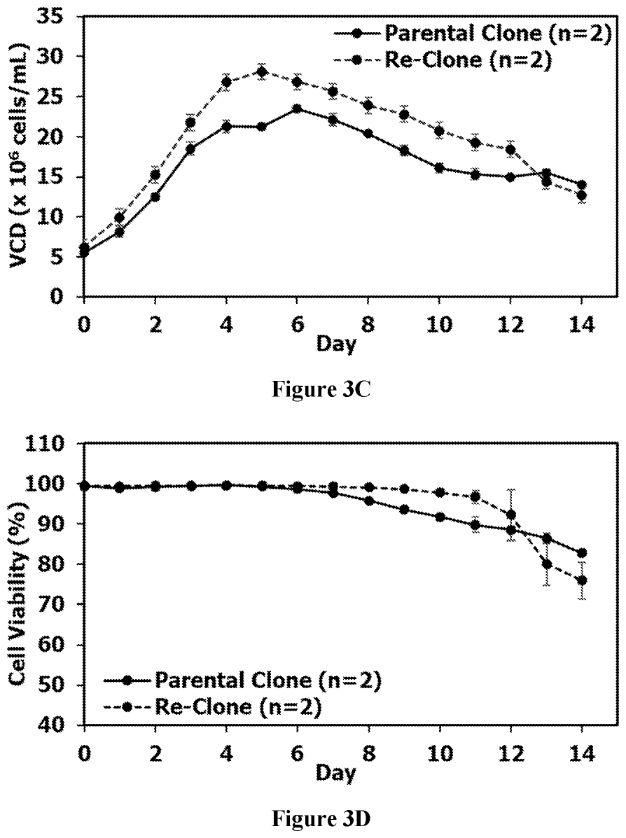

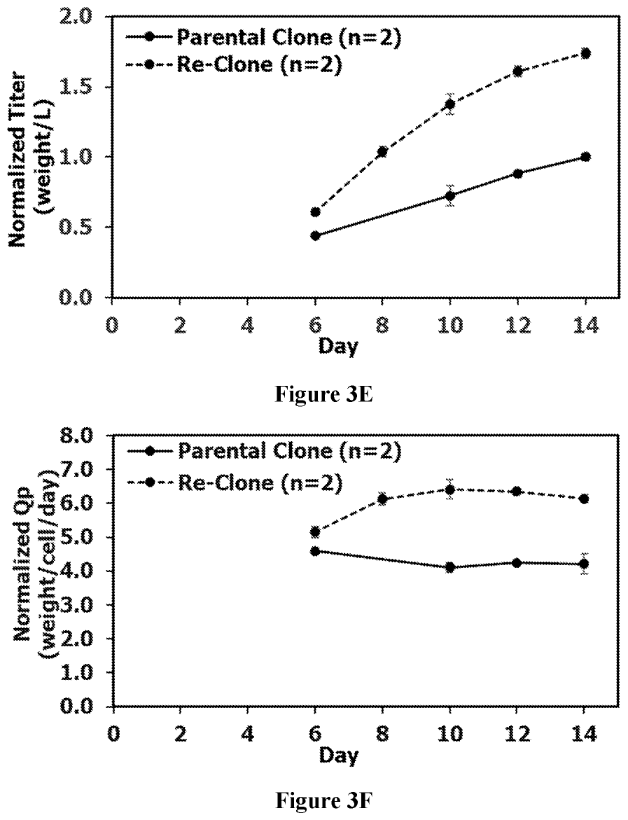

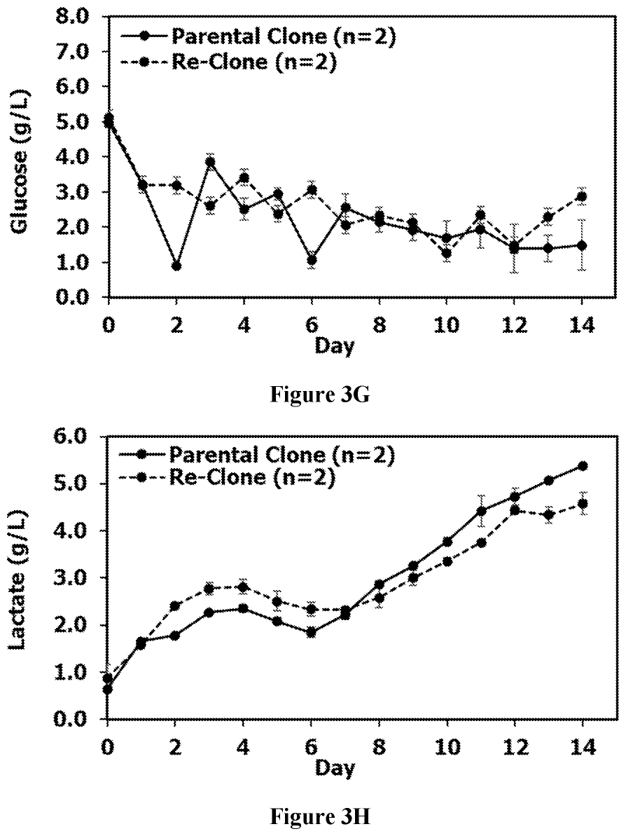

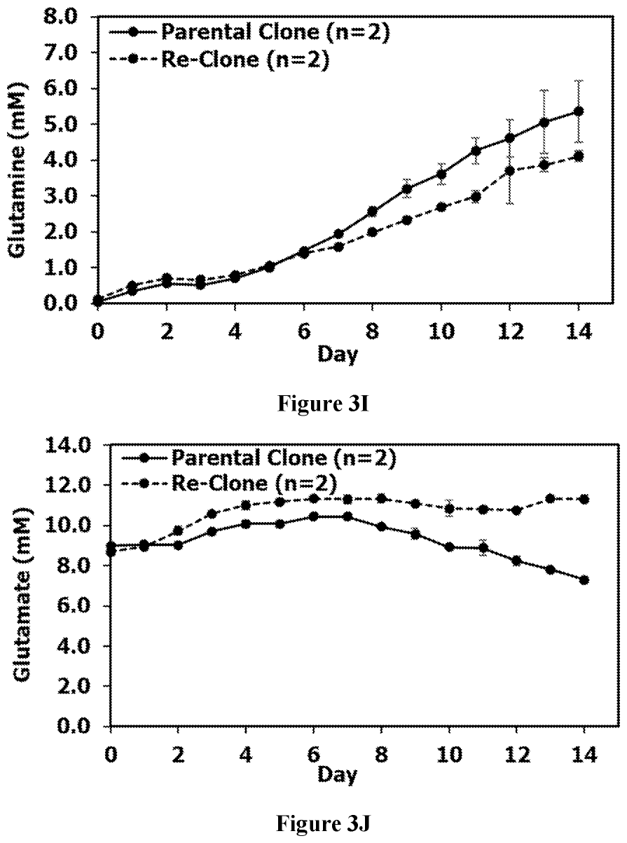

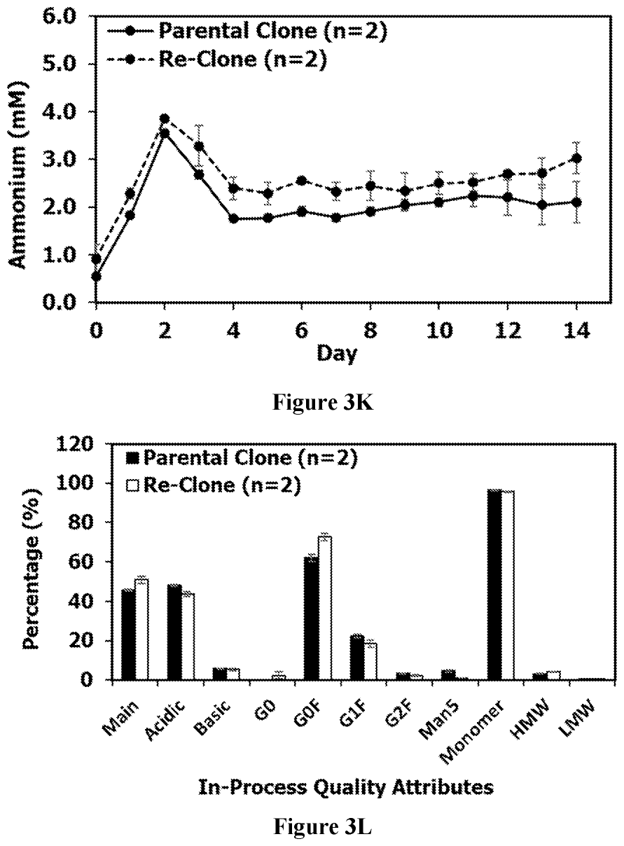

FIG. 2 and FIG. 3 show a comparison between mAb1 parental clone and lead re-clone (as re-clone) culture performance in shake flasks, N−1 fed-batch 5-L bioreactors and production fed-batch 5-L bioreactors. The average doubling time of the mAb1 re-clone was 23.4±1.8 hours, while the average doubling time of its parental clone was 22.9±2.4 hours in seven passages in shake flasks from the vial thaw (FIG. 2A). The N−1 fed-batch cultures were performed in 5-L bioreactors with a target seeding density of 1.1×10⁶ cells/mL and a duration of 4 days. The re-clone N−1 fed-batch seed culture started at an initial VCD of 1.2×10⁶ cells/mL and reached a final (i.e., day 5) VCD of 21.4×10⁶ cells/mL, leading to an average doubling time of 27.0 hours. In comparison, the parental clone started at an initial VCD of 0.9±0.0×10⁶ cells/mL and reached a final (day 5) VCD of 21.1±0.0×10⁶ cells/mL, with an average doubling time of 26.8±0.3 hours (FIG. 2B and FIG. 3A). Both clones maintained high viabilities (i.e., above 95%) on day 5 (FIG. 3B). Overall, seed train and N−1 cell growth for the mAb1 re-clone were comparable to the mAb1 parental clone.

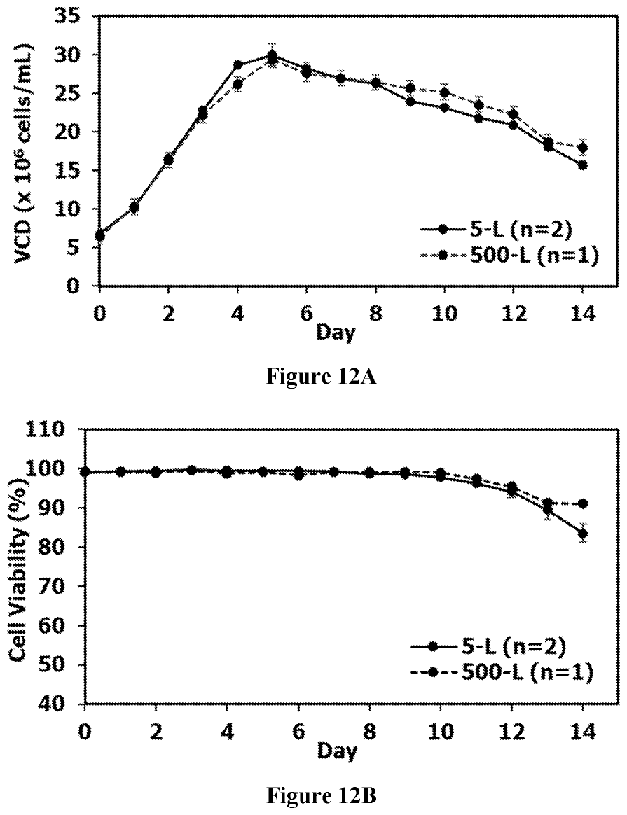

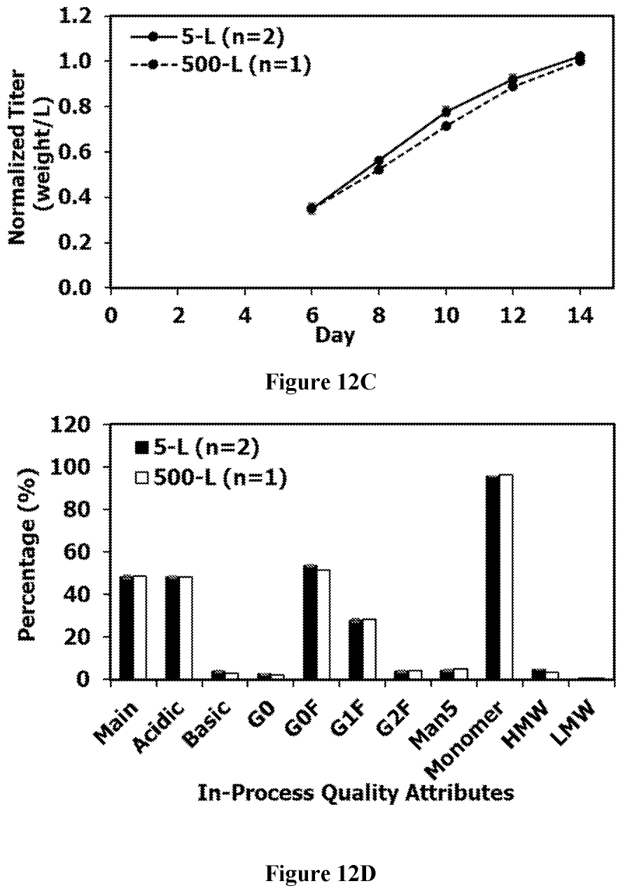

Production fed-batch cultures were performed in 5-L bioreactors with a seeding density of 6.0×10⁶ cells/mL and a duration of 14 days. Total feed amounts were 53% of initial volume, but feeding started on day 2 for the parental clone and on day 1 for the re-clone. In addition, the parental clone was fed once per day while the re-clone was fed twice per day. Re-clone production cultures reached a peak VCD of 28.1±0.1×10⁶ cells/mL on day 5, a final VCD of 12.7±0.7×10⁶ cells/mL and a final viability of 76.0%±4.6%; in comparison, the parental clone reached a peak VCD of 23.5±0.3×10⁶ cells/mL on day 6, a final VCD of 14.0±0.3×10⁶ cells/mL and a final viability of 82.8%±0.6% (FIG. 3C-3D). The re-clone condition achieved a significantly higher day 14 normalized titer of 1.7±0.04 (normalized to the day 14 titer achieved by the parental clone; FIG. 3E), and a 46% higher day 14 normalized Qp of 6.1±0.1 weight/cell/day compared with the day 14 normalized Qp of 4.2±0.2 weight/cell/day for the parental clone (FIG. 3F). The re-clone cultures had similar glucose and lactate profiles, but lower glutamine levels and higher glutamate and ammonium levels towards the end of the production culture when compared with the parental clone (FIG. 3G-3K). The re-clone cultures had comparable quality attributes in terms of charge variants, SEC and N-glycan profiles except a higher GOF level when compared with the parental clone (FIG. 3L). mAb1 re-clone higher productivity performance was scaled up to the 500-L scale. These results demonstrated the mAb1 re-cloning successfully increased the titer by 70% and cell specific productivity by 46% while maintaining comparable product quality attributes (FIG. 3L and Table 3). mAb1 re-clone performance was successfully scaled up to the 500-L scale in the pilot plant. VCD, viability, titer, and product quality were similar between the two scales (FIG. 12A-12D).

TABLE 3
Potency for the parental and re-clone normalized to the reference
standard for each mAb. The re-clones passed the potency release tests,
and potency values were similar between the parental clone and re-clone
for mAb1 and mAb2 (parental clone data is not available for mAb3).
ELISA potency assay was used for mAb1 (parental clone n = 3, re-
clone n = 1) and mAb3 (re-clone n = 3). A cell-based potency assay was
used for mAb2 (parental clone n = 1, re-clone n = 1).

| | Clone | Potency (%) |
|---|---|---|
| mAb1 | Parental Clone | 102.7 ± 10.8 |
| | Re-clone | 94.8 |
| mAb2 | Parental Clone | 94.0 |
| | Re-clone | 91.8 |
| mAb3 | Parental Clone | N/A |
| | Re-clone | 104.0 ± 2.0 |

### Example 3

Re-Clone Process Development for mAb2 Production

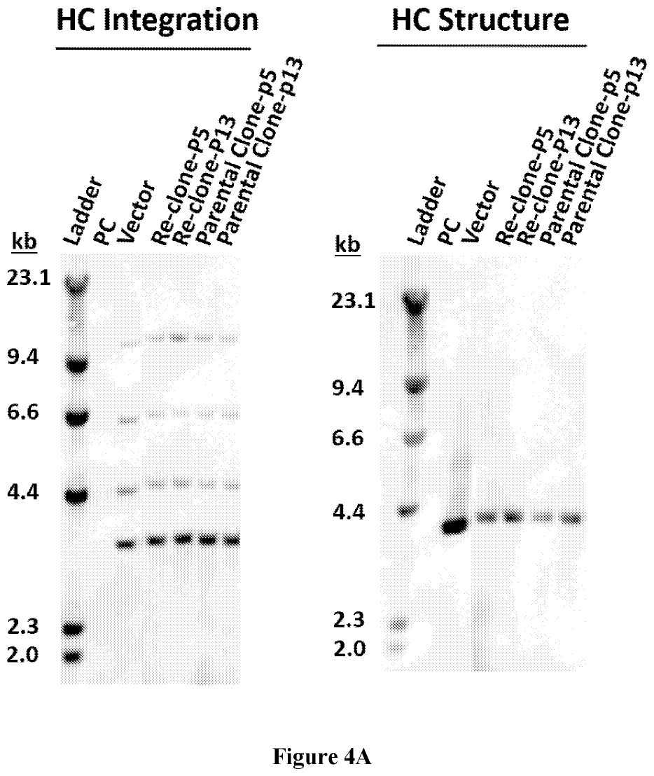

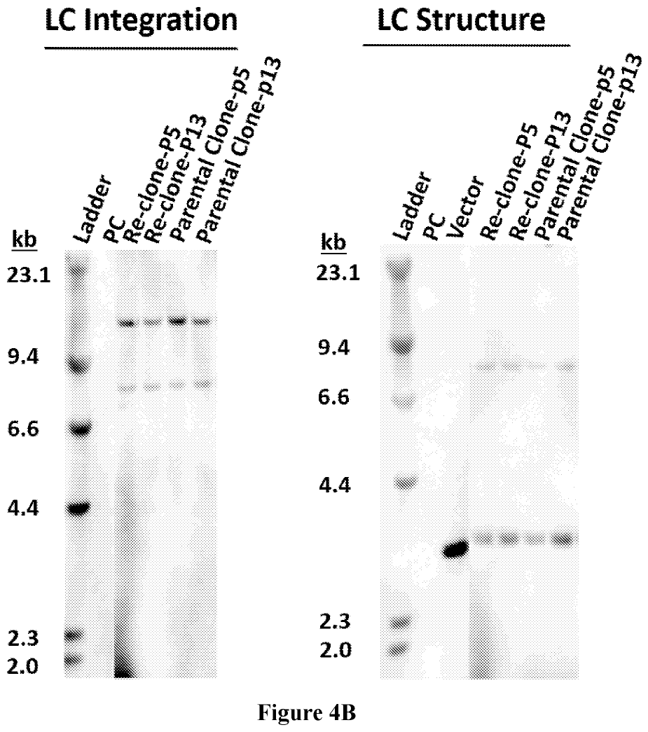

In order to increase productivity, re-cloning was performed for mAb2 using a similar strategy as that used for mAb1. A RCB vial of parental mAb2 was thawed in seed media with 1×MSX, cultured for one passage and then cultured for eight passages in seed media with 4×MSX before single cell sorting and RCB generation. The lead re-clone and the parental clone for mAb2 had the same mAb gene sequence, the same target gene integration and structure profiles (FIG. 4A and FIG. 4B), and similar heavy chain (HC) and light chain (LC) gene copy numbers (GCN) at approximately WCB age (Table 1).

Compared to the parental clone with the same conditions for mAb2, the fed-batch titer for the re-clone increased by 25% in 50-mL TubeSpin bioreactors (Table 2), indicating that the re-cloning strategy did increase titer other than just increasing MSX concentrations in seed media alone for mAb2.

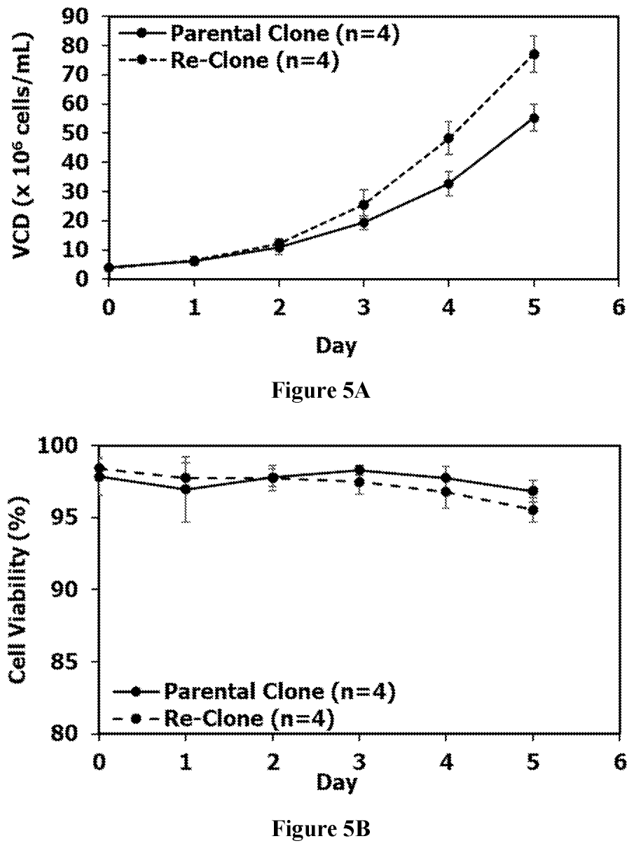

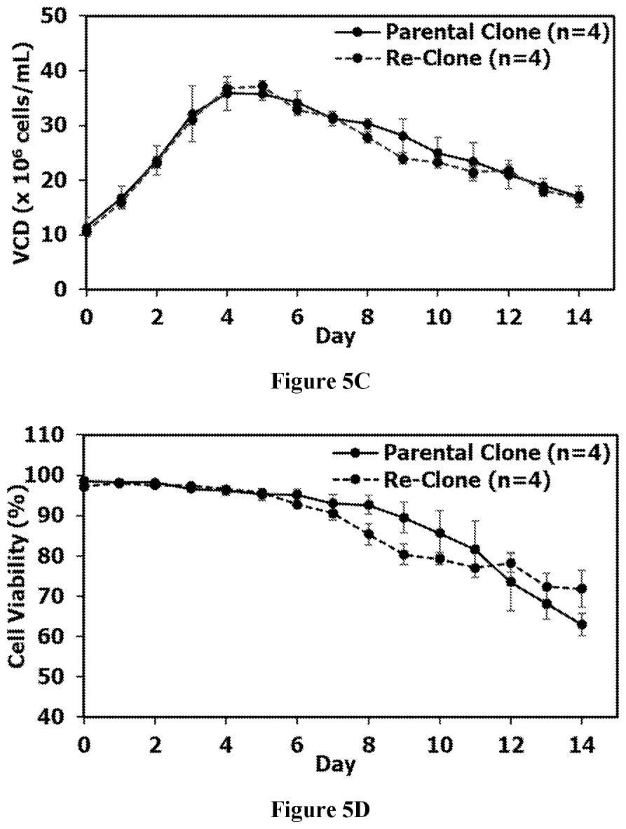

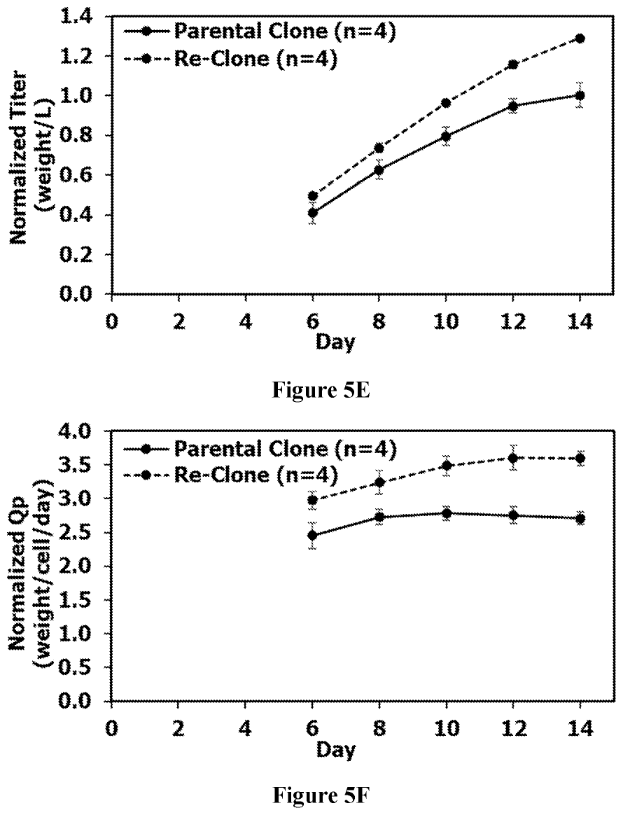

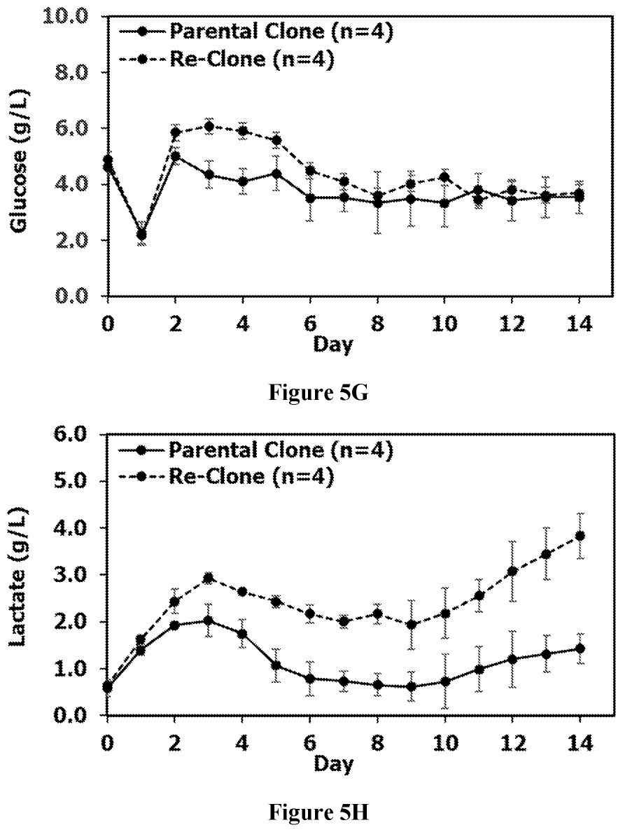

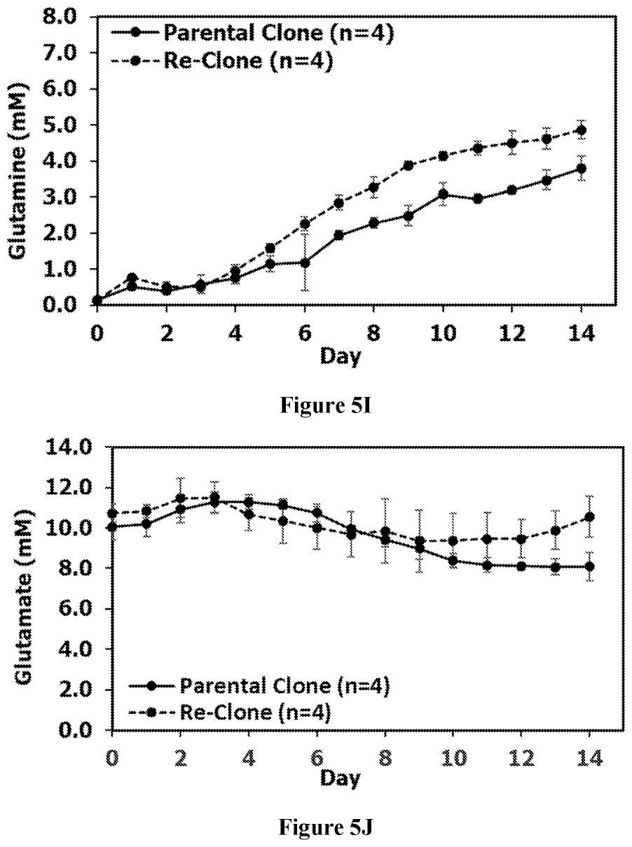

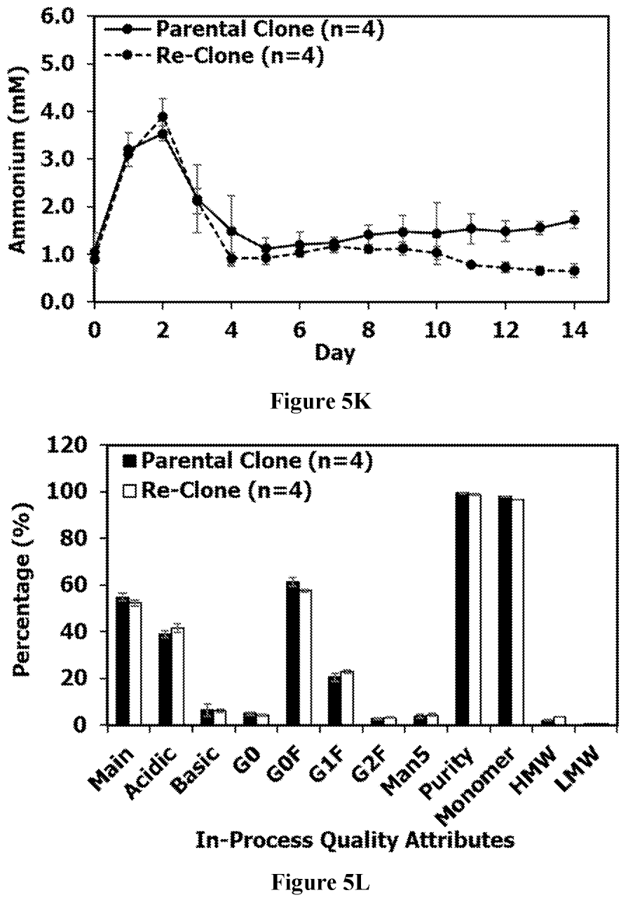

FIG. 2 and FIG. 5 show a comparison between the mAb2 parental clone and lead re-clone culture performance in shake flasks, N−1 perfusion 5-L bioreactors and production 5-L fed-batch bioreactors. The average doubling time of the mAb2 re-clone was 26.6±0.8 hours, while the average doubling time of the parental clone was 34.8±1.5 hours in shake flasks. Thus, mAb2 re-clone doubling time was 20% lower than its parental clone in seed culture prior to the N−1 culture (FIG. 2A). N−1 perfusion cultures were performed in 5-L bioreactors with a target seeding density of 4×10⁶ cells/mL and a duration of 5 days. The perfusion seed culture for the re-clone reached a higher day 5 VCD of 77.1±6.2×10⁶ cells/mL with a faster average doubling time of 27.4±1.6 hours when compared to the parental clone, which reached a day 5 VCD of 55.2±4.6×10⁶ cells/mL with an average doubling time of 31.4±1.2 hours (FIG. 2B and FIG. 5A). Both clones maintained high viabilities (i.e., above 95%) on day 5 (FIG. 5B).

Production fed-batch cultures were performed in 5-L bioreactors with a target seeding density of 10×10⁶ cells/mL and a duration of 14 days. Total feed volume was optimized for the parental clone (45%) and for the re-clone (60%), and each clone is shown in the figures in its best representative condition. Note that the optimized condition for the parental clone (i.e., 45% feed volume) was tested for the re-clone and led to lower titer, while a higher feed volume than 45% was tested for the parental clone and also led to lower titer; therefore, the re-clone for mAb2 increased the design space in which the upstream process was able to be optimized for increased titer. Re-clone production cultures reached a peak VCD of 37.2±1.6×10⁶ cells/mL on day 5, a final VCD of 16.8±1.6×10⁶ cells/mL and a final viability of 71.8%±4.6%. In comparison, the parental clone achieved a peak VCD of 35.8±1.2×10⁶ cells/mL on day 4, a final VCD of 17.1±2.0×10⁶ cells/mL and a final viability of 63.0%±2.7% (FIGS. 5C and 5D). The re-clone process resulted in a significantly higher day 14 normalized titer of 1.3±0.01 (normalized to the parental clone day 14 titer) and a 30% higher day 14 normalized Qp of 3.6±0.11 weight/cell/day compared with the day 14 normalized Qp of 2.7±0.09 weight/cell/day for the parental clone (FIGS. 5E and 5F). The re-clone cultures had similar glucose and glutamate profiles (FIGS. 5G and 5J), higher concentrations of glutamine and lactate (FIG. 5H and 5I), and lower concentrations of ammonium in the late stages of the production culture (FIG. 5K) when compared with the parental clone metabolic profiles. The different metabolic profiles for mAb2 parental and re-clone were partially due to the different feed volumes. Both clones had comparable quality attributes in terms of charge variants, SEC and N-glycan profiles (FIG. 5L). These results suggested the mAb2 re-cloning enabled process improvements that increased the volumetric productivity by 30% and the specific productivity (Qp) by 30% while maintaining comparable quality attributes (FIG. 5L and Table 3).

### Example 4

Re-Clone Process Development for mAb3 Production

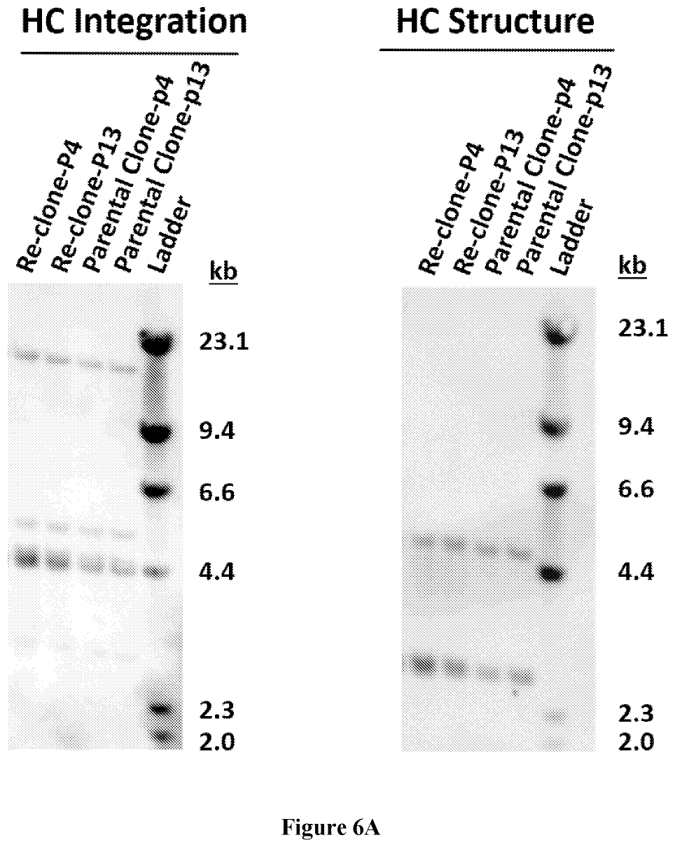

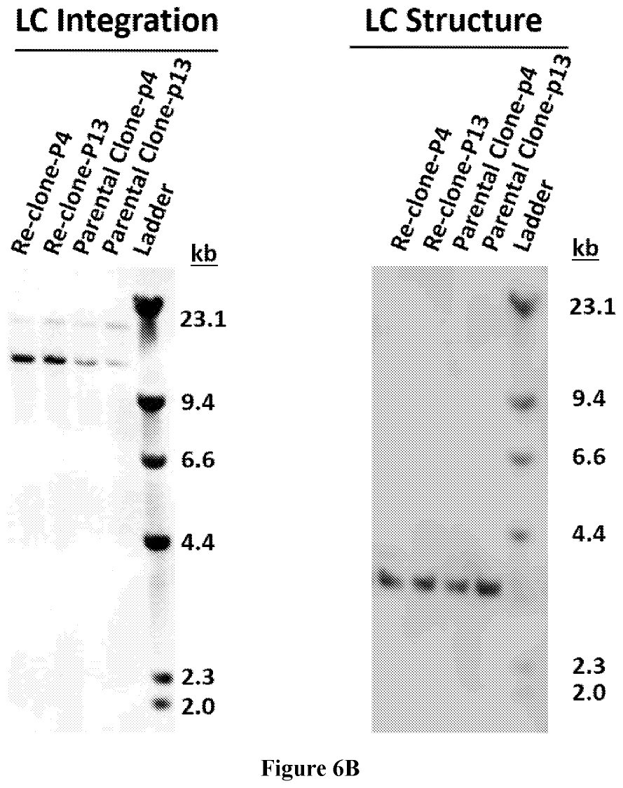

In order to increase mAb3 productivity, a similar re-cloning strategy was applied to mAb3 during late phase process development from a RCB vial of the mAb3 parental clone. Like mAb1 and mAb2, the lead re-clone for mAb3 and the parental clone for mAb3 had the same mAb gene sequence, the same target gene integration and structure profiles (FIG. 6A and FIG. 6B), and similar heavy chain (HC) and light chain (LC) gene copy numbers (GCN) at approximately WCB age (Table 1).

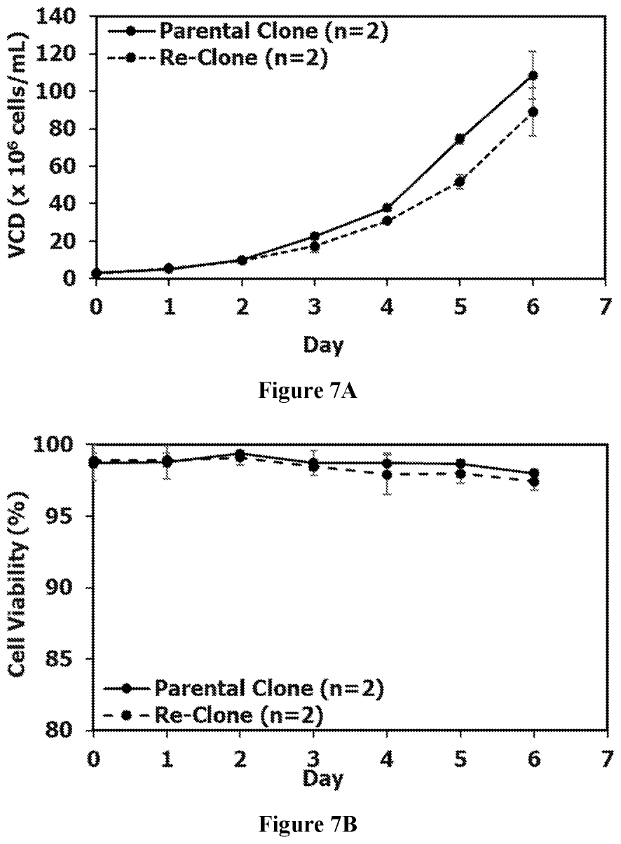

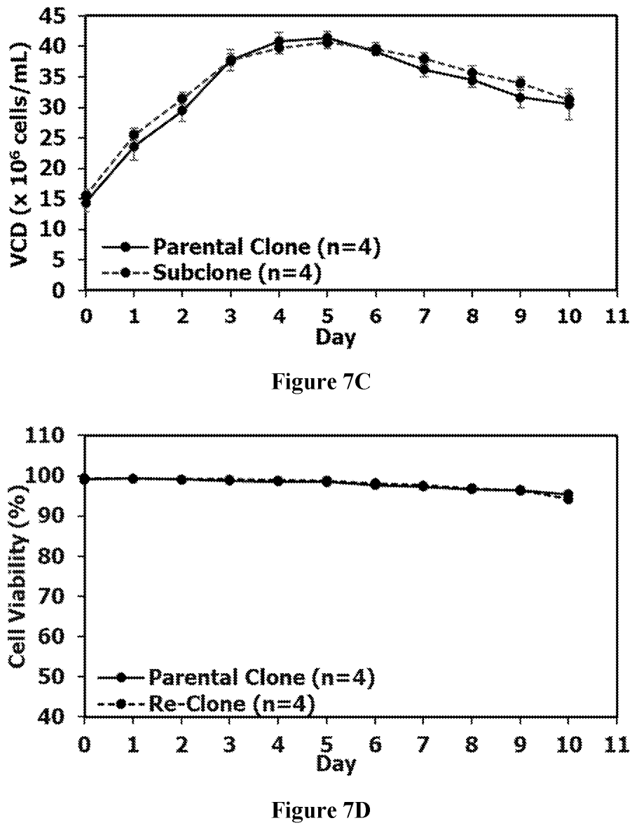

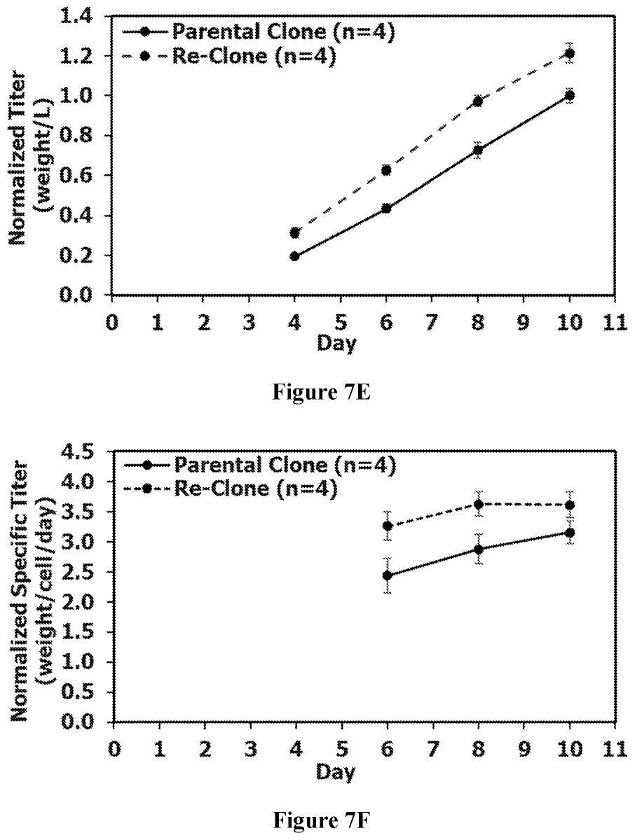

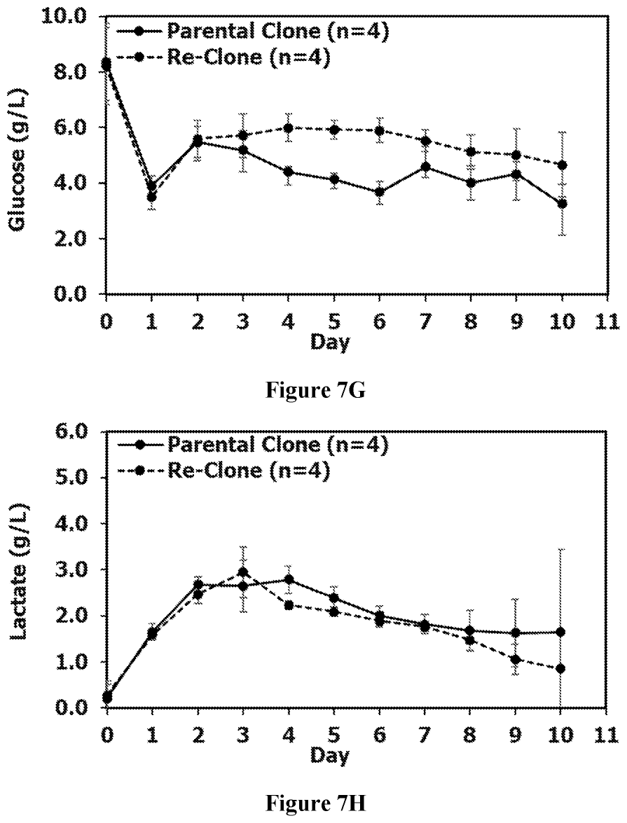

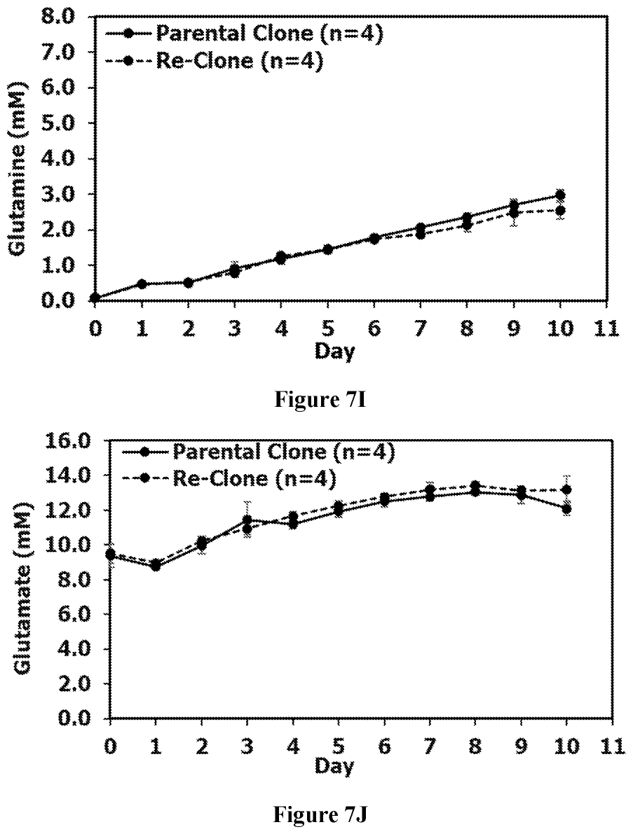

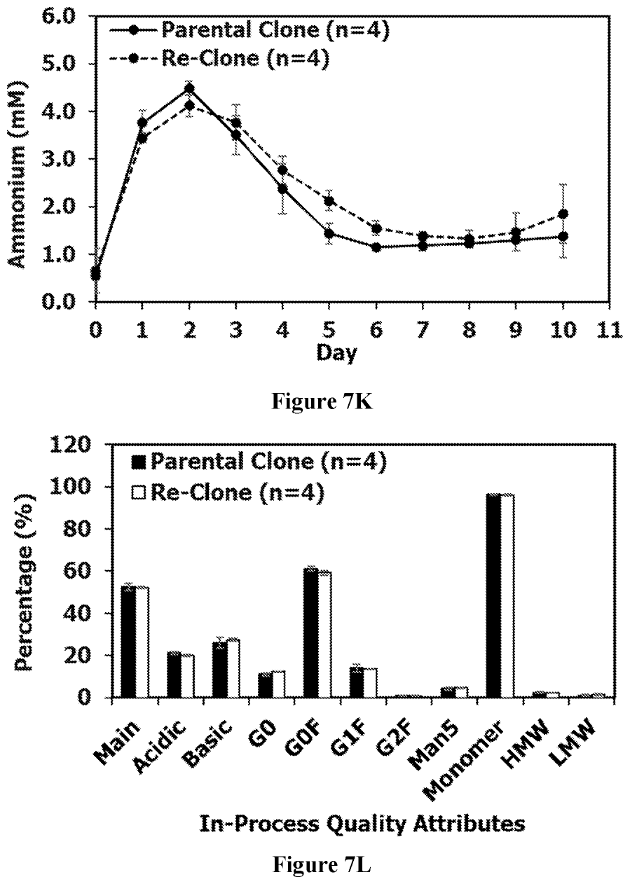

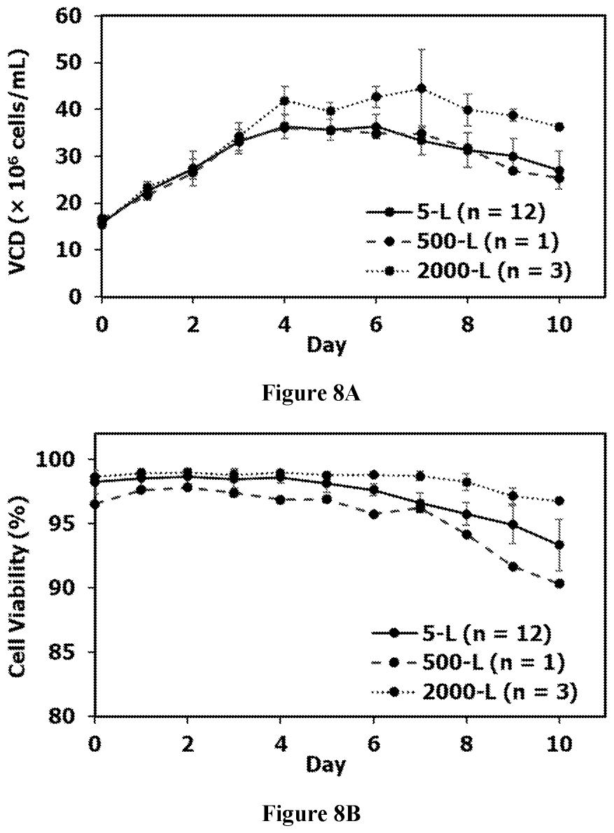

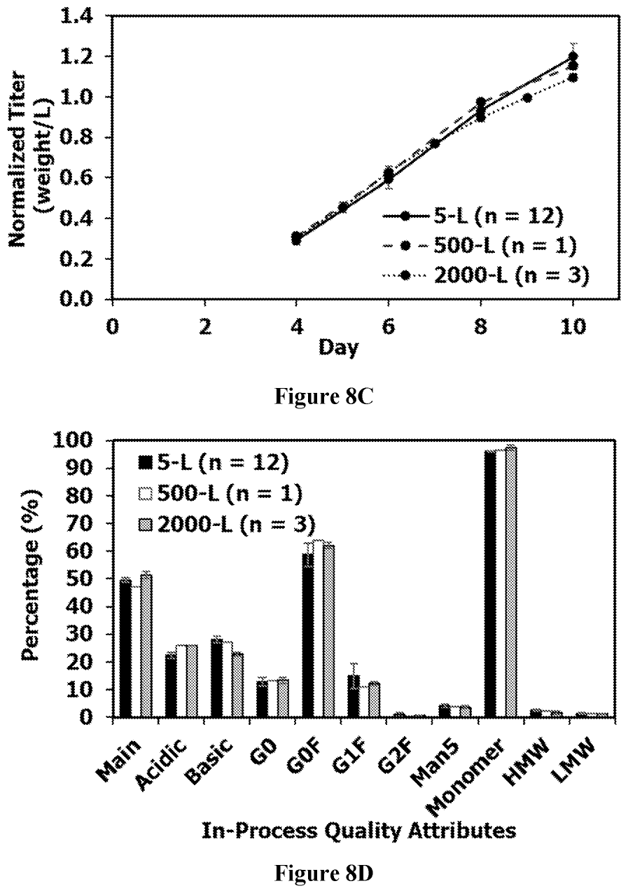

Compared to the parental clone with the seed medium containing 1×MSX in the shake flask and N−1 seed culture, mAb3 re-clone was cultured in the seed medium with 4× MSX for the seed train. The mAb3 lead re-clone cell growth average doubling time was 25.8±1.1 hours compared to the doubling time of 23.1±1.4 hours for the parental clone in shake flasks for the seed train (FIG. 2A). The N−1 perfusion cultures were performed in 5-L bioreactors with a target seeding density of 3×10⁶ cells/mL and a duration of 6 days. The re-clone N−1 perfusion seed culture reached a final (i.e., day 6) VCD of 88.9=13.0×10⁶ cells/mL with an average doubling time of 28.9±1.3 hours; in comparison, the parental clone achieved a day 6 VCD of 108.5±12.6×10⁶ cells/mL with an average doubling time of 27.1±0.4 hours (FIG. 2B and FIG. 7A). Both clones maintained high viabilities (i.e., above 95%) on day 6 (FIG. 7B). The production fed-batch cultures were performed at 5-L bioreactors with a target seeding density of 15×10⁶ cells/mL and a duration of 10 days. The re-clone production cultures reached a peak VCD of 40.6±2.3×10⁶ cells/mL on day 5, a final VCD of 31.3±1.5×10⁶ cells/mL and a final viability of 94.2%±0.7% on day 10. The parental clone production culture reached a peak VCD of 41.4±1.1×10⁶ cells/mL on day 5, a final VCD of 30.6±2.5×10⁶ cells/mL and a final viability of 95.4%±0.6% on day 10 (FIGS. 7C and 7D). The re-clone resulted in a higher day 10 normalized titer of 1.2±0.05 (normalized to the parental clone day 10 titer; FIG. 7E), and a 12.5% higher day 10 normalized Qp of 3.6±0.2 weight/cell/day compared with the day 10 normalized Qp of 3.2±0.2 weight/cell/day for the parental clone (FIG. 7F). The re-clone cultures had similar glutamine, glutamate, lactate and ammonium profiles to the parental clone; however, the glucose was typically at higher concentrations for the re-clone (FIG. 7G-7K). The parental and re-clone had comparable quality attributes in terms of charge variants, SEC and N-glycan profiles (FIG. 7L). These results demonstrated mAb3 re-cloning successfully increased the productivity by 20% with comparable cell growth and quality attributes (FIG. 7L and Table 3). mAb3 re-clone performance was successfully scaled up from the 5-L bioreactor scale to the 2000-L bioreactor in the clinical manufacturing facility (FIG. 8A-8D).

### Example 5

Re-Clone Process Development for mAb4 Production

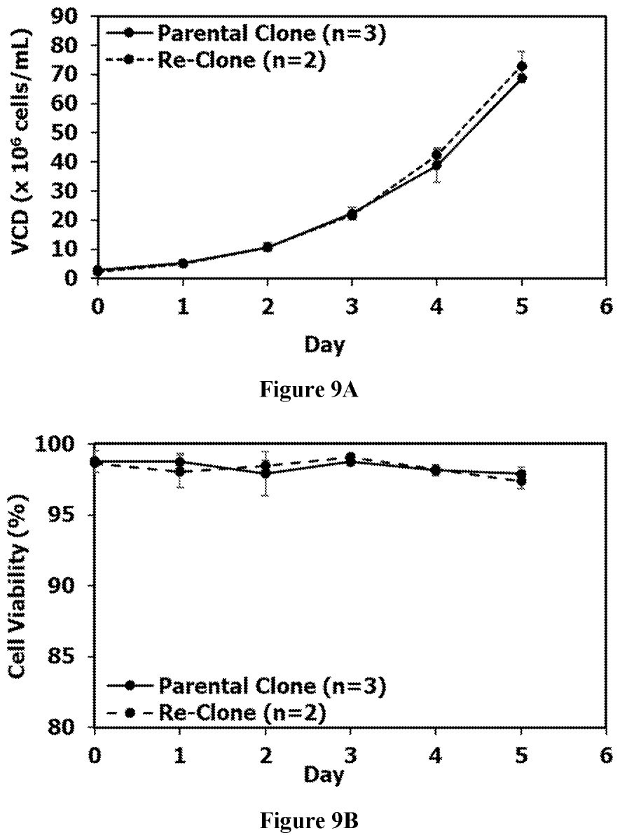

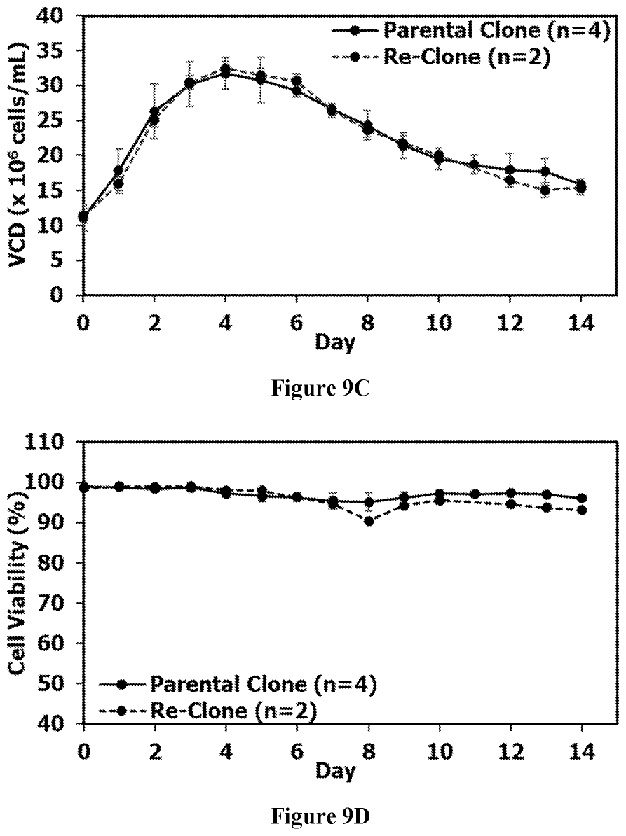

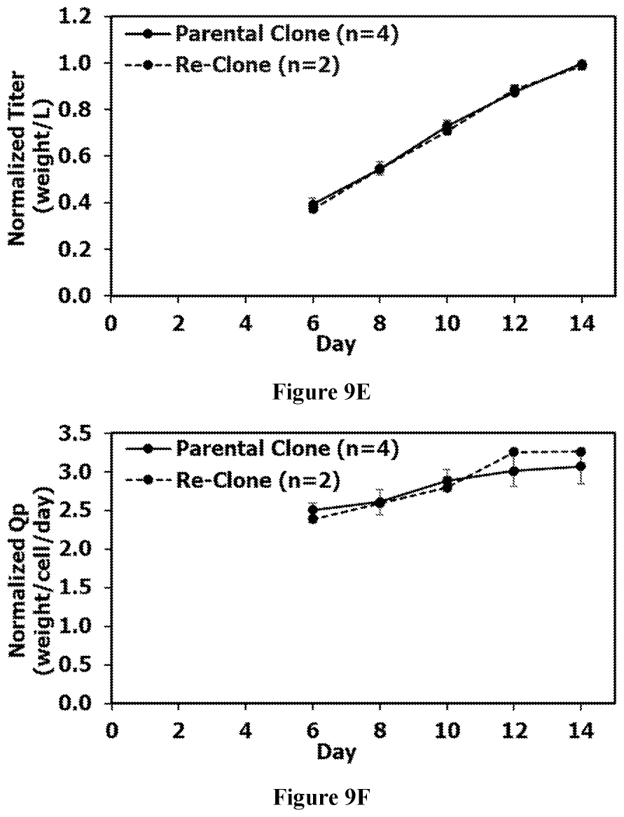

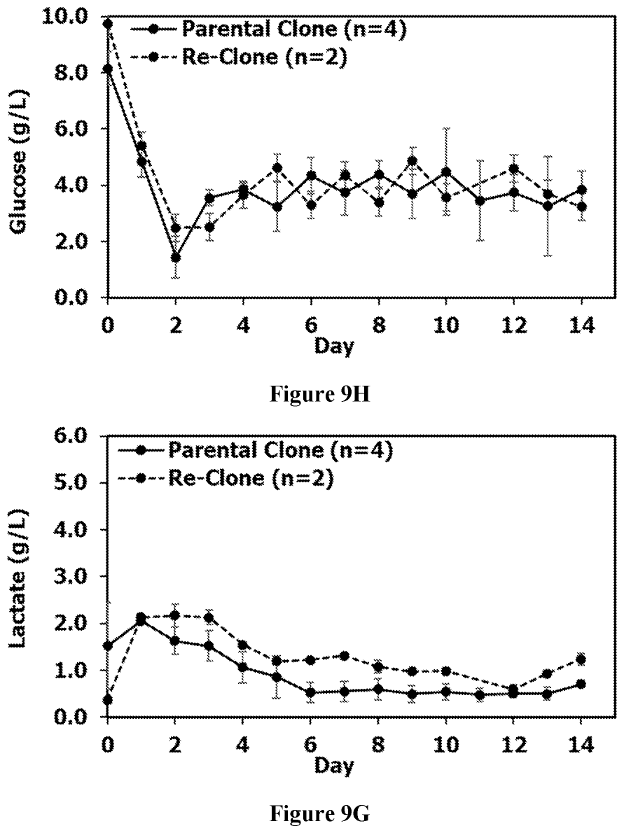

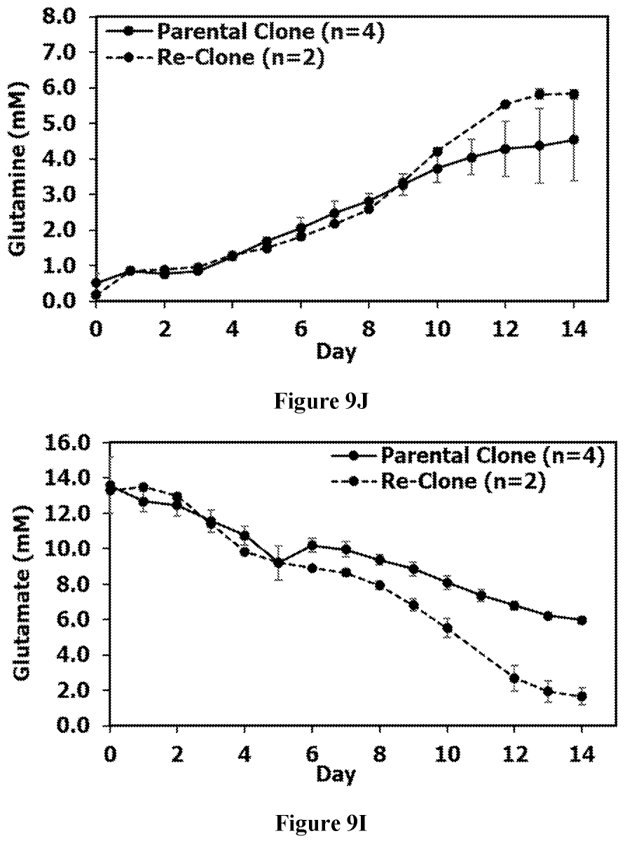

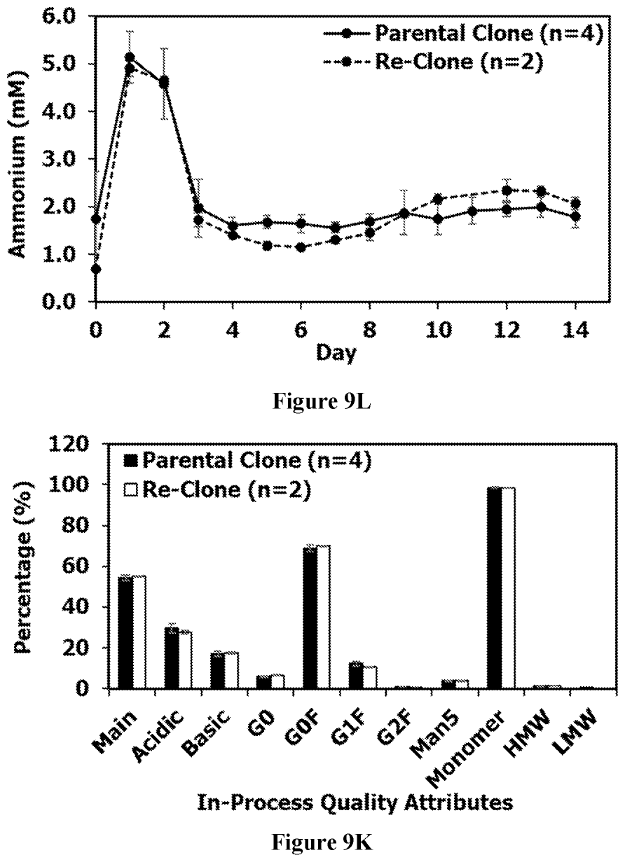

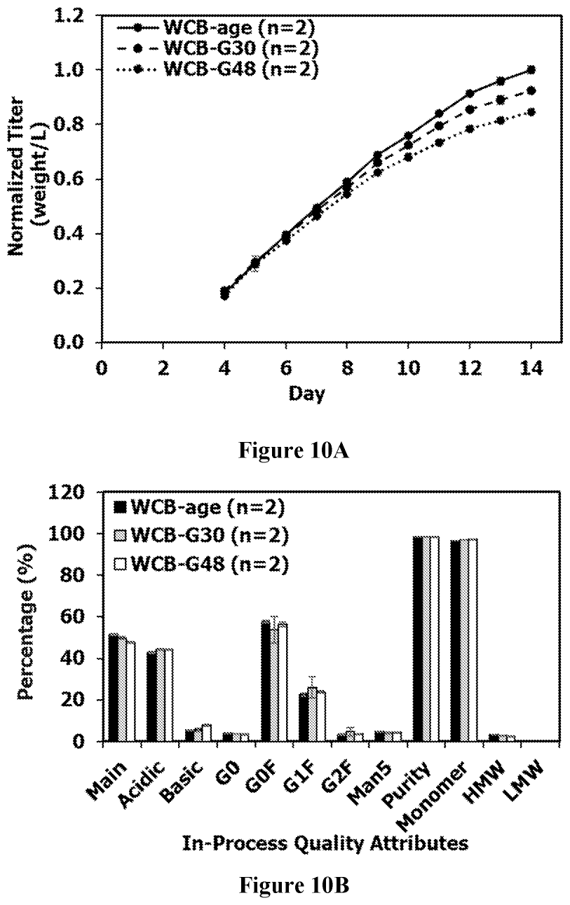

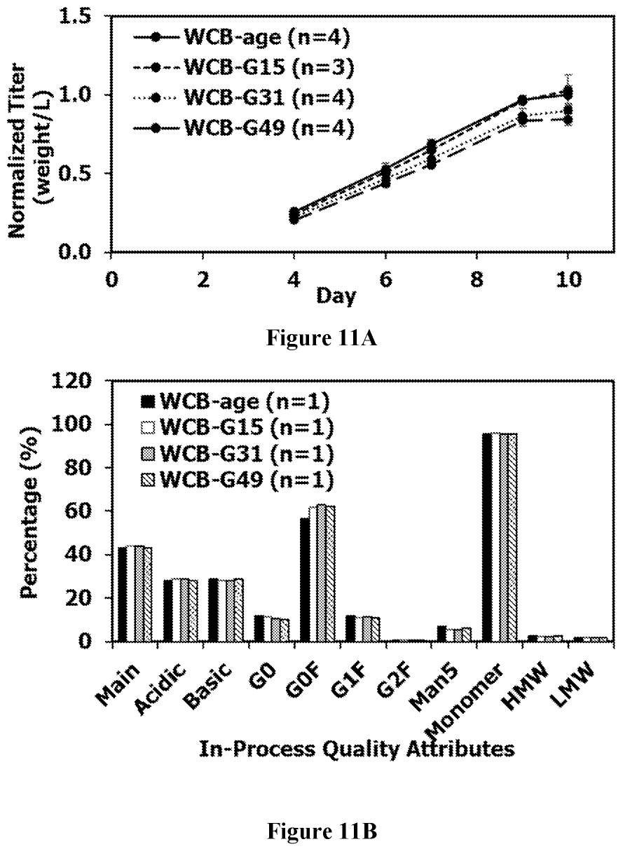

Re-cloning was also performed in an attempt to improve mAb4 productivity. A MCB vial of the parental mAb4 was thawed and cultured in the seed media with 1×MSX before single cell sorting since increasing MSX concentration from 1× to 4× did not show a positive effect on titer in mAb4 early process development. A top re-clone was selected with standard re-cloning procedures. The average doubling time of the mAb4 re-clone in the seed train was 24.5±1.7 hours, which was slightly slower than the parental clone which had a doubling time of 21.7±0.2 hours in shake flasks (FIG. 2A). The N−1 perfusion cultures were performed in 5-L bioreactors with a target seeding density of 2.5×10⁶ cells/mL and a duration of 5 days. The re-clone N−1 perfusion seed culture reached a day 5 VCD of 73.0±4.8×10⁶ cells/mL with an average doubling time of 23.8±0.3 hours, compared with the parental clone which achieved a day 5 VCD of 68.7±1.4×10⁶ cells/mL and an average doubling time of 26.5±0.7 hours (FIGS. 2B and 9A). The parental clone and re-clone each had high viabilities (i.e., above 95%) on day 5 (FIG. 9B).

Production fed-batch cultures were performed in 5-L bioreactors with a target seeding density of 10×10⁶ cells/mL and a duration of 14 days. The re-clone had similar cell growth profiles to the parental clone. The re-clone production cultures reached a peak VCD of 32.4±0.9×10⁶ cells/mL on day 4, a final VCD of 15.4±0.5×10⁶ cells/mL and a final viability of 93.1%±0.4% on day 14; the parental clone reached a peak VCD of 31.7±2.3×10⁶ cells/mL on day 4, a final VCD of 15.9±0.8×10⁶ cells/mL and a final viability of 96.0%±0.6% on day 14 (FIGS. 9C and 9D). The mAb4 re-clone cultures resulted in a similar normalized titer and Qp when compared with the normalized titer and Qp of the parental clone at day 14 (FIGS. 9E and 9F). The re-clone cultures had similar glucose, lactate and ammonium profiles but higher glutamine levels and lower glutamate levels in the late stages of culture compared with the parental clone (FIG. 9G-9K). The mAb4 parental and re-clone had comparable quality attributes in terms of charge variants, SEC and N-glycan profiles (FIG. 9L).

1. A method of producing a recombinant polypeptide of interest, comprising:
a) culturing a monoclonal cell line under optimized culture conditions;
b) re-cloning the single cell of the monoclonal cell line;
c) selecting the re-cloned cells with higher titer; and
d) culturing the selected re-cloned cells, thereby producing the recombinant polypeptide of interest.

2. A method of increasing titer of a recombinant polypeptide of interest, comprising:
a) culturing a monoclonal cell line under optimized culture conditions;
b) re-cloning the single cell of the monoclonal cell line;
c) selecting the re-cloned cells with higher titer; and
d) culturing the selected re-cloned cells, thereby producing the recombinant polypeptide of interest.

3. The method of claim 1, wherein the monoclonal cell in step a) has been cloned and selected with a selection agent, and the re-cloning in step b) is in presence of the same selection agent as used in step a).

4. The method of claim 3, wherein the selection agent concentration used in step b) is higher than the selection agent concentration used in step a).

5. The method of claim 3, wherein the selection agent is methionine sulfoximine (MSX).

6. The method of claim 5, wherein the MSX concentration in re-cloning is at least about 1 μM, at least about 2 μM, at least about 5 μM, at least about 10 μM, at least about 15 μM, at least about 20 μM, at least about 25 μM, at least about 30 μM, at least about 40 μM, at least about 50 μM, at least about 100 μM, or at least about 200 μM.

7. The method of claim 6, wherein the MSX concentration is between about 25 μM to about 75 μM.

8. The method of claim 1, wherein the selected re-cloned cells are cultured in a bioreactor.

9. The method of claim 8, wherein the bioreactor is at least 5 L, at least 10 L, at least 50 L, at least 100 L, at least 200 L, at least 500 L, at least 1,000 L, at least 2,000 L, at least 5,000 L, at least 10,000 L, at least 15,000 L, at least 20,000 L or at least 25,000 L.

10. The method of claim 1, wherein the monoclonal cells are from a research cell bank (RCB), a master cell bank (MCB) or a working cell bank (WCB).

11. The method of claim 1, wherein the cells are mammalian cells.

12. The method of claim 11, wherein the mammalian cells are selected from the group consisting of CHO, VERO, BHK, HEK, HeLa, COS, MDCK and hybridoma cells.

13. The method of claim 11, wherein the cells are CHO cells.

14. The method of claim 13, wherein the CHO cells are CHO glutamine synthetase (GS) knockout (GS−/−) cell line

15. The method of claim 1, wherein the recombinant polypeptide of interest is an antibody or antigen-binding fragment.

16. The method of claim 15, wherein the antibody is an mAb.

17. The method of claim 15, wherein the antibody or antigen-binding fragment binds an antigen selected from the group consisting of PD-1, PD-L1, CTLA-4, LAG-3, TIGIT, TIM-3, GITR, CXCR4, CD73, HER2, VEGF, CD20, CD40, CD11a, tissue factor (TF), PSCA, IL-8, IL-13, and SARS-COV-2 spike protein.

18. The method of claim 2, wherein the monoclonal cell in step a) has been cloned and selected with a selection agent, and the re-cloning in step b) is in presence of the same selection agent as used in step a).

19. The method of claim 2, wherein the selected re-cloned cells are cultured in a bioreactor.

20. The method of claim 2, wherein the monoclonal cells are from a research cell bank (RCB), a master cell bank (MCB) or a working cell bank (WCB).
